(19) United States
(12) Patent Application Publication
(10) Pub. No.: US 2026/0009092 A1
(43) Pub. Date: Jan. 8, 2026

(54) VIRUS DETECTION VIA PROGRAMABLE TYPE III-A CRISPR-CAS SYSTEMS AND METHODS

(71) Applicant: The Florida State University Research Foundation, Inc., Tallahassee, FL (US)

(72) Inventors: Hong Li, Tallahassee, FL (US); Sagar Sridhara, Tallahassee, FL (US); Hemant N Goswami, Tallahassee, FL (US); Charlisa Whyms, Tallahassee, FL (US)

(21) Appl. No.: 19/239,264

(22) Filed: Jun. 16, 2025

Related U.S. Application Data
(62) Division of application No. 17/709,571, filed on Mar. 31, 2022.
(60) Provisional application No. 63/168,475, filed on Mar. 31, 2021.

(51) Int. Cl. C12Q 1/70; C12Q 1/34

(52) U.S. Cl. CPC C12Q 1/70; C12Q 1/34

(57) ABSTRACT

Methods and systems, which use a reconstituted Type III-A CRISPR-Cas system, MORIARTY (Multipronged, One-pot, RNA Induced, Augmentable, Rapid, Test sYstem) for the detection of disease are provided herein. The methods and systems may be performed either without amplification or coupled to RNA transcription as one-pot reactions. The systems and methods herein may be highly sensitive and may be used to detect viruses, including SARS-CoV-2.

### CROSS-REFERENCE TO RELATED APPLICATIONS

This application is a divisional of and claims priority to U.S. application Ser. No. 17/709,571, filed Mar. 31, 2022, which claims priority to U.S. Provisional Application No. 63/168,475, filed Mar. 31, 2021, both of which are incorporated herein by reference.

### SEQUENCE LISTING

The instant application contains a Sequence Listing which has been submitted electronically in XML file format and is hereby incorporated by reference in its entirety. Said XML copy, created on Sep. 23, 2025, is named 19585-0586 SL.xml and is 50,771 bytes in size.

### BACKGROUND

Human infectious diseases constitute a broad class of diseases caused by microorganisms such as bacteria and viruses transmitted via food, air, body fluids and physical contact. These diseases can be highly contagious, and can broadly affect human health. Historically, several outbreaks namely Spanish flu and Swine flu caused by H1N1 influenza virus, Acquired Immunodeficiency Syndrome (AIDS) caused by Human Immunodeficiency virus (HIV), Zika disease caused by the Zika virus, Ebola Virus Disease (EVD) caused by the Ebola virus, Severe Acute Respiratory Syndrome (SARS) caused by SARS coronavirus (SARS-CoV) and Middle East Respiratory Syndrome (MERS) caused by MERS coronavirus (MERS-CoV) have wreaked havoc on the general population and had widespread economic implications in the world.

The global outbreak of Coronavirus disease 2019 (COVID-19) caused by a novel coronavirus named severe acute respiratory syndrome coronavirus 2 (SARS-CoV-2) is the most recent public health emergency, transmitting from human to human at very high pace across countries. The infection can either be asymptomatic or associated with pathogenesis ranging from mild, heterogenous symptoms such as fever, headache, sore throat, nausea and shortness of breath to severe respiratory failure causing death. The emergence of genetic variants of COVID-19 across the world has, in some instances, further increased the risk of mortality. One of the challenges posed by this pandemic and previous infectious diseases has been the availability of rapid testing of the virus. Other factors ranging from ambiguity in diagnosis to financial limitations associated with kits available in the market add to the plethora of challenges posed by infectious diseases.

To effectively address the pandemic and other infectious diseases, nucleic acid detection systems should be simple, rapid, specific, sensitive, cost-effective, and/or high throughput, preferably instrument-free for resource-limited settings. In this regard, several point-of-care devices/technologies such as microfluidics-based platforms, surface-plasmon-resonance-based platforms, smartphone-based detection system and lensless digital holographic imaging-based platforms have emerged. Some of the microfluidics-based platforms harness enzymatic functionality for detection purposes. Among these, Clustered, Regularly Interspaced, Short Palindromic Repeat (CRISPR) and CRISPR-Associated (Cas) proteins have been harnessed in nucleic acid detection owing to both their programmability and RNA-induced enzymatic activities. CRISPR-based technologies can take advantage of isothermal nucleic acid amplification methods such as reverse transcription loop-mediated isothermal amplification (RT-LAMP) and reverse transcription recombinase polymerase amplification (RT-RPA) without the need of thermocyclers, thereby offering convenient instrument-free solutions to testing. CRISPR-derived detection methods typically offer improved sensitivity comparable with that of PCR-based methods.

Current CRISPR-Cas based diagnostic tools/technologies employ Class 2 CRISPR-Cas effector molecules, mainly Cas12 and Cas13, but also Cas14 that possess viral DNA or RNA stimulated collateral DNase or RNase activities (Wang, M., R. Zhang, and J. Li, CRISPR cas systems redefine nucleic acid detection: Principles and methods. Biosens Bioelectron, 2020. 165: p. 112430). The Cas13-based methods such as Specific High-sensitivity Enzymatic Reporter un-LOCKing (SHERLOCK), SHERLOCK version 2 (SHERLOCKv2), Heating Unextracted Diagnostic Samples to Obliterate Nuclease (HUDSON) detect amplified viral RNA by monitoring cleaved RNA probe (Gootenberg, J. S., et al., Nucleic acid detection with CRISPR-Cas13a/C2c2. Science, 2017. 356(6336): p. 438-442). The Cas12-based methods such as DNA Endonuclease Targeted CRISPR Trans Reporter (DETECTR), one-HOur Low-cost Multipurpose highly Efficient System (HOLMES), HOLMES version 2 (HOLMESv2), All-In-One Dual CRISPR-Cas12a (AIOD-CRISPR) [37], Cas12a-Linked Beam Unlocking Reaction (CALIBURN) on the other hand, detect amplified viral DNA by monitoring cleaved DNA probe (Chen, J. S., et al., CRISPR-Cas12a target binding unleashes indiscriminate single-stranded DNase activity. Science, 2018. 360(6387): p. 436-439). However, these methods are limited.

The Type III-A CRISPR-Cas system, or Csm, is a viral RNA or transcription activated ribonucleoprotein enzyme system that comprises four enzymatic activities: (1) specific cleavage of the viral transcript by the Csm3 subunit, (2) collateral DNase by the HD domain of the Csm1 subunit, (3) cyclic oligoadenylate (cOA) synthesis by the Csm1 GGDD motif (SEQ ID NO: 1), and (4) cOA-activated collateral RNase by the ancillary enzyme Csm6 (Jia, N., et al., Type III-A CRISPR-Cas Csm Complexes: Assembly, Periodic RNA Cleavage, DNase Activity Regulation, and Autoimmunity. Mol Cell, 2019. 73(2): p. 264-277 e5). Simultaneous detection of the collateral DNase and RNase activities can offer a unique opportunity for Type III-A CRISPR-Cas systems to be repurposed for multipronged virus detection. Furthermore, the inherent responses of Csm to either viral RNA or its transcription makes the Csm-based detection suitable for both RNA and DNA viruses. However, it can be more challenging to realize the multicomponent Type III-A systems as an off-the-shelf virus detection tool than their single-subunit counterparts. Thus, a Type III-A CRISPR-Cas has not previously been repurposed for disease detection likely owing, at least in part, to the complex enzyme reconstitution process and functionality. Accordingly, improved viral detection methods, including those utilizing a Type III-A CRISPR-Cas system are highly desirable.

### BRIEF SUMMARY

Provided herein are virus detection methods, systems, and diagnostics, which, in some embodiments, are based on an in vivo-reconstituted Type III-A system, MORIARTY (Multipronged, One-pot, RNA Induced, Augmentable, Rapid, Test sYstem). MORIARTY can harness the viral RNA- or transcription-activated dual nucleic acid cleavage activities and, in some embodiments, can conveniently and advantageously detect viruses either without amplification or coupled to RNA transcription as one-pot reactions. Embodiments of the methods provided herein are highly sensitive.

In embodiments, the methods of detection provided herein comprise providing a sample comprising a virus, wherein the virus comprises a cognate target RNA; contacting the sample with an effector complex and an ancillary protein, wherein the effector complex binds to the cognate target RNA, and the binding of the effector complex to the cognate target RNA produces at least one messenger comprising a cyclic oligoadenylate, wherein the at least one messenger activates nonspecific ssRNA cleavage activity in the ancillary protein to produce detectable DNase activity, detectable RNase activity, or a combination thereof, and analyzing the sample with one or more reporters to detect the detectable DNase activity, detectable RNase activity, or a combination thereof.

In embodiments, systems for detecting a virus are provided herein comprising a L1Csm effector complex, a L1Csm6 protein, a first reporter comprising an RNA oligo flanked by a first fluorophore-quencher pair; and a second reporter comprising a DNA oligo flanked by a second fluorophore-quencher pair.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is understood that both the foregoing general description and the following detailed description are exemplary and explanatory only and are not restrictive.

### BRIEF SUMMARY OF THE DRAWINGS

FIG. 1 is a schematic representation of an expression-plasmid, according to one embodiment. FIG. 1 discloses SEQ ID NOS 1, 3, 4, 3, and 5, respectively, in order of appearance.

FIGS. 2A-2F depicts reaction components for amplification detection, according to one embodiment. FIG. 2A is a schematic representation of reaction components in a single pot. FIGS. 2B-2D show a simultaneous rise in DNA-Alexa and RNA-FAM fluorescence under three different conditions tested: 10 mM MnCl₂, 0 mM ATP (FIG. 2B), 10 mM MnCl₂, 0.05 mM ATP (FIG. 2C) and 10 mM MgCl₂, 0.5 mM ATP (FIG. 2D). FIGS. 2E-2F show the augmentability of MORIARTY by combined use of 1 or 0.5 M DNA-FAM and 1 or 0.5 M RNA-FAM as probes under two different conditions tested: 10 mM MnCl₂, 0.05 mM ATP (FIG. 2E), 10 mM MgCl₂, 0.5 mM ATP (FIG. 2F).

FIGS. 3A-3F depict MORIARTY reaction components for transcription coupled detection, according to one embodiment. FIG. 3A is a schematic representation of reaction components in a single pot. FIGS. 3B-3D show the multiprongness of T7 MORIARTY by simultaneous rise in DNA-Alexa and RNA-FAM fluorescence under two different conditions tested: 17 mM MgCl₂, 0.5 mM ATP, 0 mM MnCl₂ (FIGS. 3B-3C), and 17 mM MgCl₂, 0.5 mM ATP, 0.5 mM MnCl₂ (FIG. 3D). FIGS. 3E-3F show combined use of 1 or 0.5 M DNA-FAM and 1 or 0.5 M RNA-FAM under two different conditions tested: 17 mM MgCl₂, 0.5 mM ATP, 0 mM MnCl₂ and 1 nM L1Csm6 (FIG. 3E), 17 mM MgCl₂, 0.5 mM ATP, 0.5 mM MnCl₂ and 250 nM L1Csm6 (FIG. 3F).

FIG. 4 is a schematic representation of SARS-CoV-2 genome harboring Orf1ab, S, E, M and N genes, according to one embodiment. FIG. 4 discloses SEQ ID NOS 6 and 7, respectively, in order of appearance.

FIGS. 5A-5D depict a sensitivity of MORIARTY in amplification-free setting towards the detection of SARS-CoV-2. FIG. 5A shows the 37mer S_CTR model RNA serially diluted and tested in MORIARTY. FIG. 5B depicts the slope of the curves in FIG. 5A. FIG. 5C, shows the S_IVT_RNA samples serially diluted and tested in MORIARTY. FIG. 5D depicts the slope of the curves in FIG. 5C.

FIG. 6 is a schematic depicting a workflow of patient sample retrieval, RNA extraction, RT-RPA and T7-MORIARTY using L1Csm_S/L1Csm6 system, according to one embodiment.

FIGS. 7A-7C and 8 depict detection of SARS-CoV-2 by RT-RPA and T7-MORIARTY in patient samples, according to one embodiment. FIG. 7A is a schematic representation of reaction components. FIG. 7B shows SARS-CoV-2 control RNA samples serially diluted, added to RT-RPA reaction and tested in T7 MORIARTY. FIG. 7C depicts the slope of the curves in FIG. 7B.

FIG. 8 shows viral RNA samples extracted from patients and RT-RPA treated and tested in T7-MORIARTY.

FIGS. 9A-9B depict the effects of metal ions, ATP and L1Csm/Csm6 mutations on amplification-free MORIARTY RNase and DNase probe signals, according to one embodiment.

FIG. 9A shows the effects of increasing ATP (left to right) in the presence of Mn²⁺, Mg²⁺, or both. FIG. 9B shows the effect of L1Csm/Csm6 mutations.

FIG. 10 depicts the effects of metal ions and ATP on amplification-free MORIARTY with combined RNase and DNase probe signals, according to one embodiment.

FIGS. 11A-11D show the effects of concentrations of L1Csm6 (FIG. 11A), probe (FIG. 11B), DNA template (FIG. 11C) and supplementary Mn²⁺ (FIG. 11D) on T7-MORIARTY, according to one embodiment.

FIGS. 12A-12B depict testing of T7-MORIARTY at different RNA concentrations, according to one embodiment. Both S_IVT_RNA (FIG. 12A) and S_RNA (FIG. 12B) were used to test T7-MORIARTY.

FIGS. 13A-13C depict fluorescent data of samples tested using T7-MORIARTY, according to one embodiment. FIG. 13A shows the rise in fluorescence of samples from patients 1-6 and 12-14. FIG. 13B shows the rise in fluorescence of samples from patients 7-11. FIG. 13C is a bar diagram of the fluorescent intensities of the samples in FIGS. 13A-13B, at 40 minutes.

### DETAILED DESCRIPTION

Provided herein are methods and systems, which may use an in vivo-reconstituted Type III-A system, MORIARTY (Multipronged, One-pot, RNA Induced, Augmentable, Rapid, Test sYstem) for the detection of viruses. MORIARTY can harness the viral RNA- or transcription-activated dual nucleic acid cleavage activities and can conveniently and advantageously detect viruses either without amplification or coupled to RNA transcription as one-pot reactions. In embodiments, the systems and methods herein, may be used to detect SARS-CoV-2. In embodiments, the systems and methods herein, may be used to detect viruses other than SARS-CoV-2, including but not limited to a coronavirus (e.g., SARS/MERS), influenza virus, HIV, Ebola virus, and/or Zika Virus.

The systems and methods provided herein may be sensitive. In embodiments, the systems and methods herein have a low detection limit. For example, in embodiments the systems and methods herein provide sensitivity of less than 500 fM, such as less than 200 fM, less than 100 fM, less than 50 fM, less than 20 fM, less than 10 fM, less than 5 fM, less than 1 fM, or less than less than 0.5 fM. In embodiments, the systems and methods herein may reach 5 fM sensitivity in amplification-free and 60 copies/ml sensitivity via isothermal amplification within 30 minutes. The high sensitivity, ease in enzyme production, and/or flexible reaction conditions can make MORIARTY a highly effective and affordable detection method with broad applications.

In some embodiments, the systems herein include a Type III-A CRISPR-Cas system for virus detection employing both amplification-free and RT-RPA based strategies. MORIARTY-based detection typically is a highly-sensitive system and can have advantages over those based on qPCR without the requirement for thermocyclers. Unique to MORIARTY is its multiprongeness, which can yield cumulative signals for detection under multiple buffer conditions. For example, in a Mn²⁺ and ATP-free buffer, the viral RNA stimulated DNase can be readily detectable while in a Mg²⁺ buffer with ATP, the viral RNA-stimulated and cOA₆-mediated RNase activity typically dominates. In some embodiments, with an optimized solution containing Mn²⁺ and a low concentration of ATP, both DNase and RNase activities are simultaneously detectable in an additive manner. It is understood that other buffer conditions may be used.

In some embodiments, the methods herein include amplification-free detection with MOARITY under the Mn²⁺/low ATP condition, which can directly detect in vitro transcribed SARS-CoV-2 S gene mRNA as low as 5 fM (˜3000 copies/μL).

The enzymatic property of Csm in its activation by RNA transcription permits, in some embodiments, the construction of a one-pot detection with DNA amplified from viral RNA. Embodiments of this convenient procedure reached high sensitivity with either model SARS-CoV-2 virus or human patient samples. In some embodiments, MORIARTY-based assay results show a consistent detection sensitivity to those by qPCR and seem to have a larger dynamic range than qPCR in detecting viral RNA. Noteworthy, the temperature range of 37° C. to 42° C. for all the steps in some embodiments of the T7-MORIARTY-based detection can eliminate the requirement of expensive equipment and makes it potentially compatible with the low-cost hand warmer-mediated heating solution as demonstrated previously.

In embodiments, the methods of detection herein include a L1Csm effector complex. In embodiments, the ancillary protein comprises L1Csm6. In embodiments, the at least one messenger comprises cOA₆. In embodiments, the one or more reporters comprise one or more fluorescence reporters. In embodiments, the one or more reporters comprise (i) an RNA oligo flanked by a first fluorophore-quencher pair, and (ii) a DNA oligo flanked by a second fluorophore-quencher pair. In embodiments, the first fluorophore-quencher pair and the second fluorophore-quencher pair are the same. In embodiments, the virus comprises a spike protein, and the spike protein comprises the cognate target RNA. In embodiments, the virus is SARS-CoV-2. In embodiments, the contacting of the sample with the effector complex occurs in a liquid. In embodiments, the liquid is a buffer.

In embodiments, a concentration of the effector complex in the liquid is about 200 nM to about 300 nM, and the concentration of the ancillary protein is about 0.1 to about 3 nM. In embodiments, the methods of detection comprise detecting both the detectable DNase activity and the detectable RNase activity. In embodiments, the virus is not amplified. In embodiments, the methods herein further comprise amplifying the virus prior to the contacting step. In embodiments, the methods herein are performed in a single container.

In embodiments, the systems provided herein comprise a buffer. In embodiments, the buffer comprises Mg2+, ATP, or a combination thereof. In embodiments, the systems comprise a T7 promoter sequence. In embodiments, the system further comprises a sample comprising a virus to be detected. In embodiments, the system can detect the virus at 5 fM or less.

The systems and methods herein demonstrate that MORIARTY is a versatile virus detection method with high sensitivity. Given that the genomes of infectious pathogens are made up of either DNA or RNA, multipronged diagnostic tools such as MORIARTY can be employed towards the detection of multiple nucleic acid targets. With the availability of many known Type III-A CRISPR-Cas systems and their individual biochemical differences, MORIARTY offers a broad range of nucleic acid detections under a wide range of conditions.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein. The supplementary tables and figures are in attached Appendix A.

### Example 1—MORIARTY Simultaneously and Cumulatively Detects DNase and RNase Fluorescence

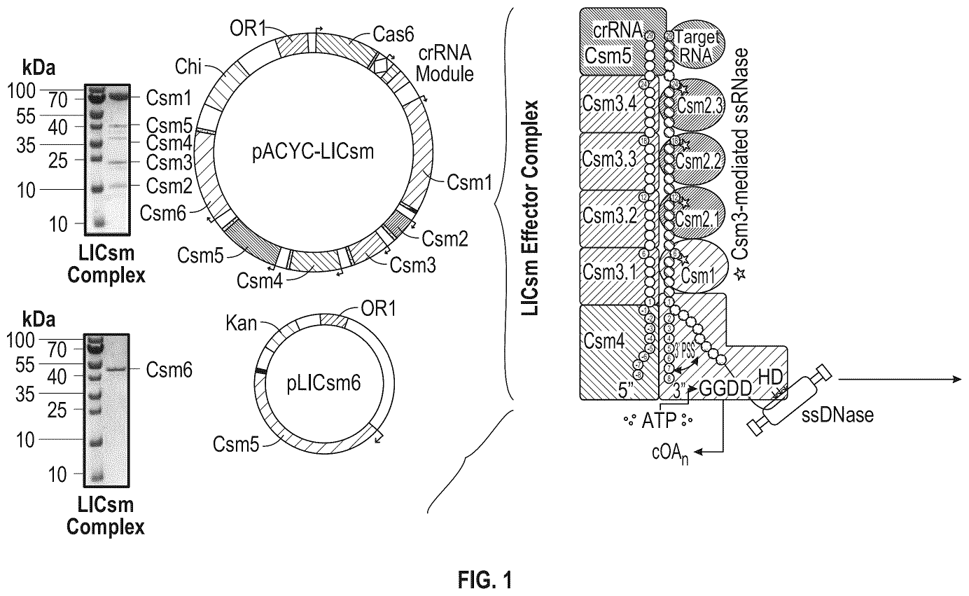

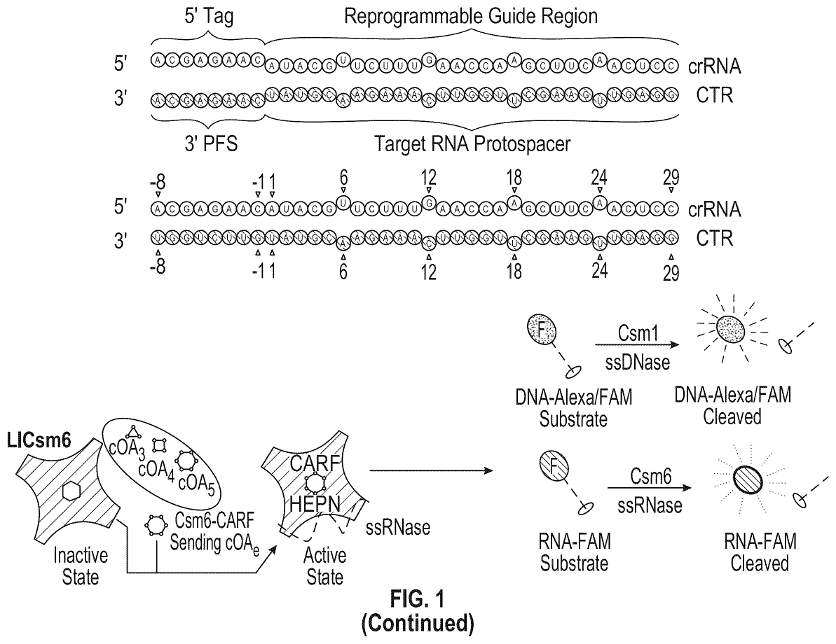

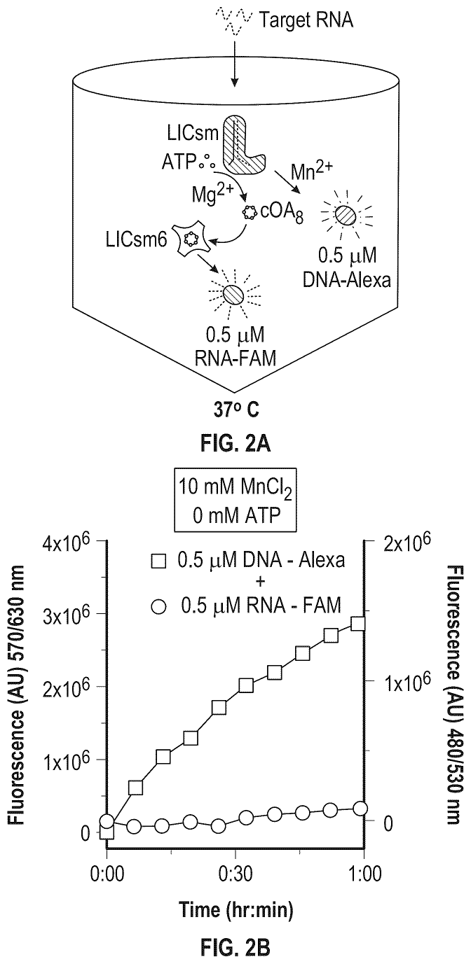

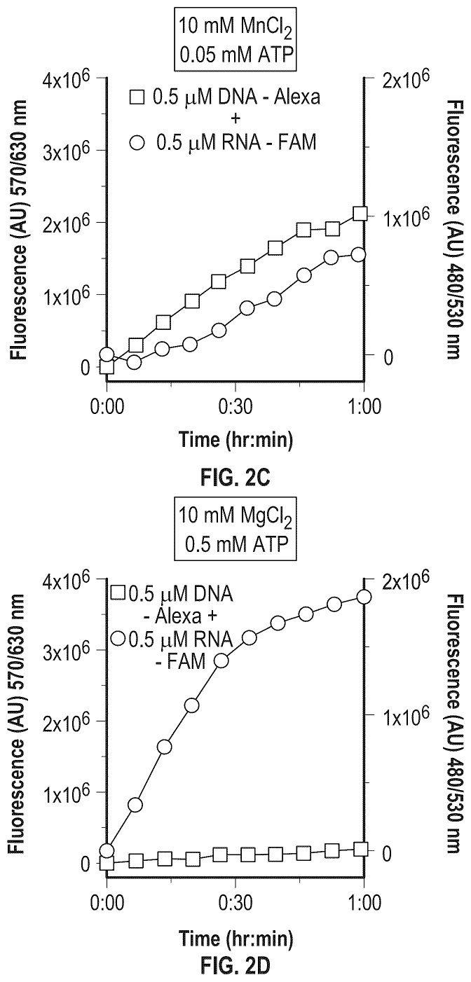

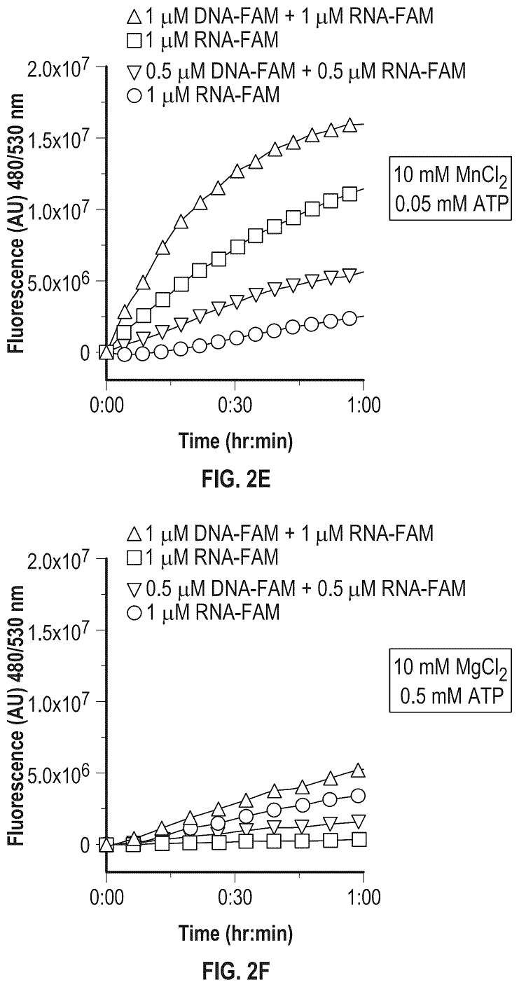

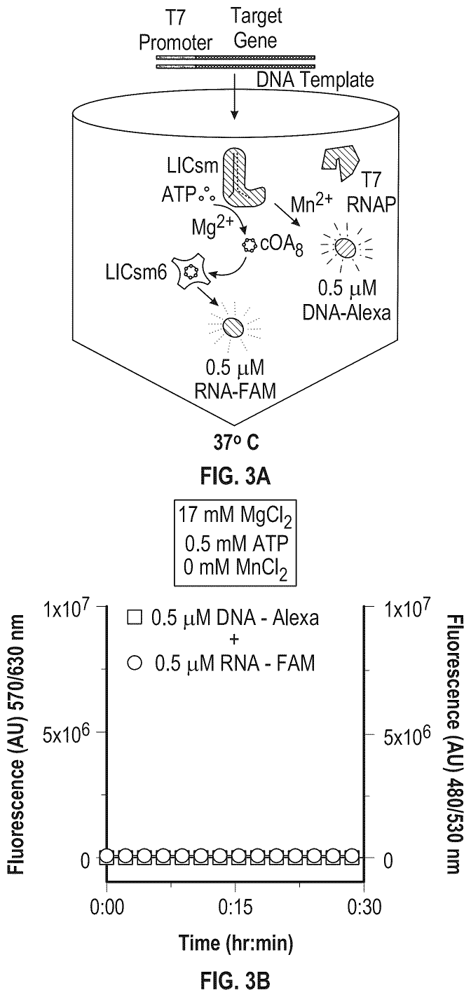

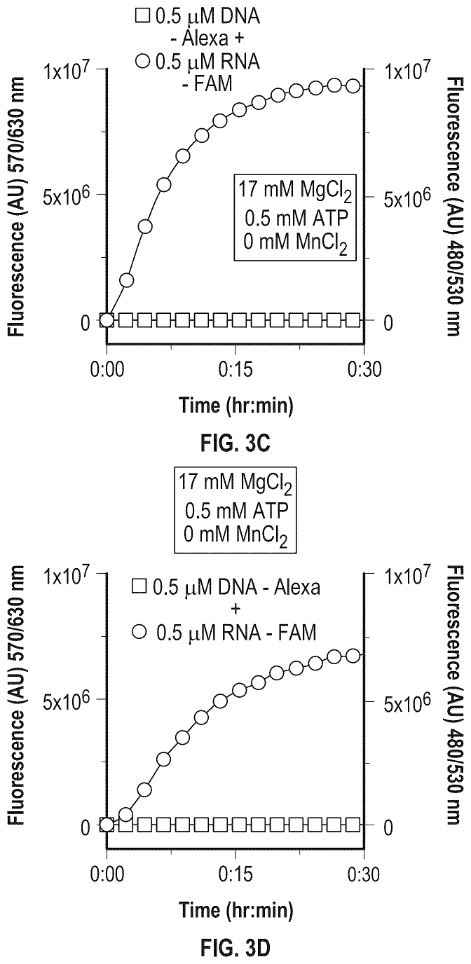

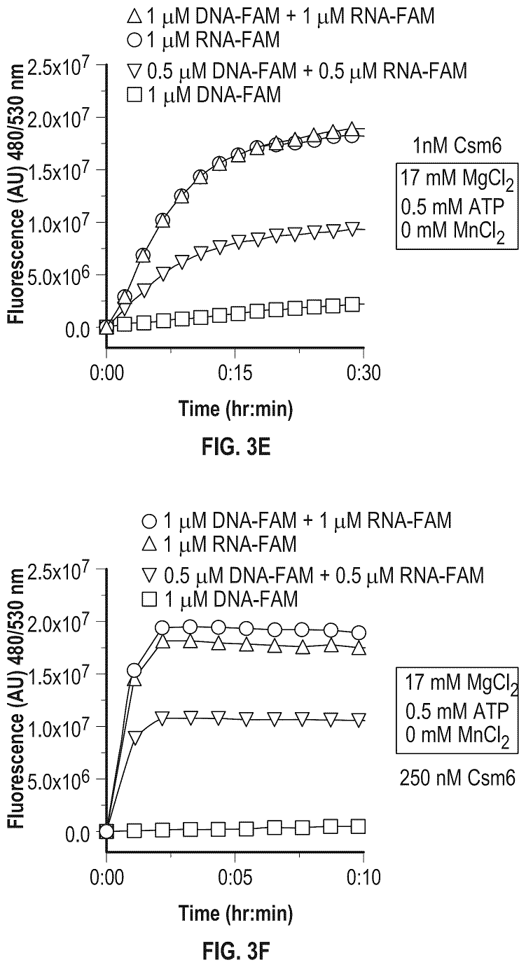

The L1Csm effector complex was produced using an all-in-one codon-optimized expression plasmid encoding all Csm subunits and modified crRNA locus (FIG. 1, FIGS. 2A-2F, and FIGS. 3A-3F). Specifically, in FIG. 1, expression-plasmids, purified Csm components, and schematic representation of the L1Csm/L1Csm6 CRISPR-Cas system. RNA induced cleavage by Csm3, DNase by Csm1, cOA₆ synthesis by Csm1 and cOA₆-stimulated RNase by Csm6 are indicated. CTR denotes cognate target RNA and NTR denotes not non-cognate target RNA. The DNA fluorescent probes (DNA-Alexa/DNA-FAM) used were substrates for L1Csm1-stimulated DNase activity. The RNA fluorescent probe (RNA-FAM) was a substrate for cOA₆ stimulated L1Csm6-mediated RNase activity. The cleavage of DNA-Alexa was indicated by the rise in fluorescence intensity at 570/630 nm excitation/emission wavelength. The cleavage of DNA-FAM/RNA-FAM was indicated by the rise in fluorescence intensity at 480/530 nm excitation/emission wavelength.

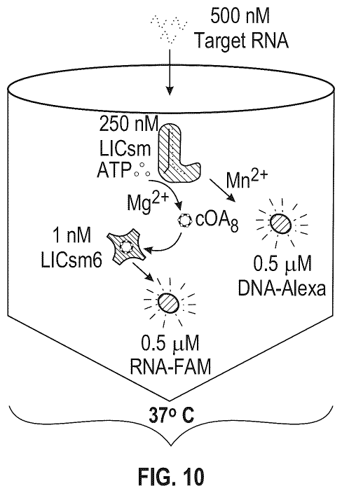

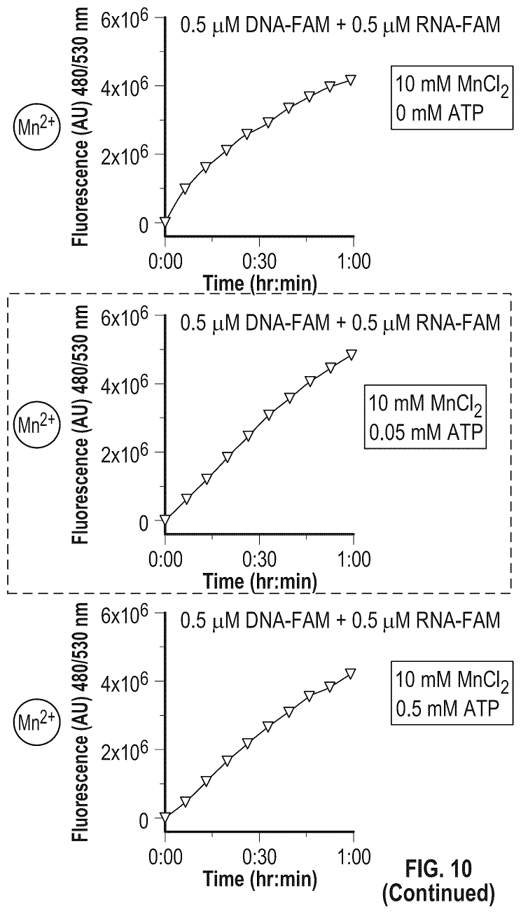

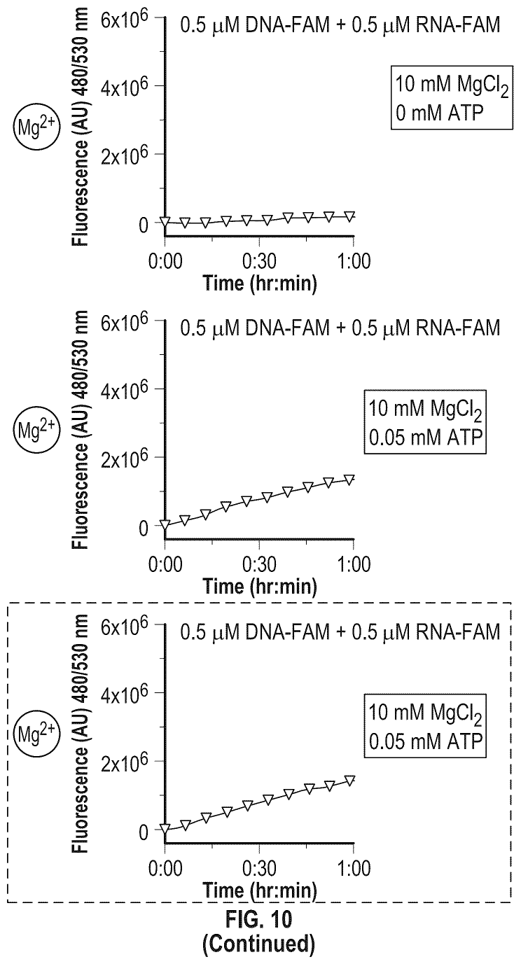

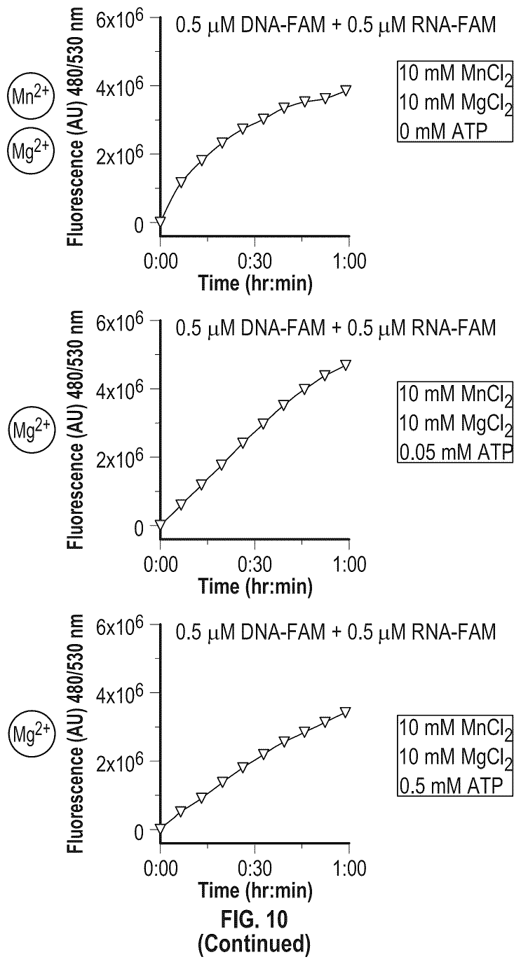

The purified L1Csm cleaved single stranded DNA (ssDNA) and polymerized Adenosine triphosphate (ATP) to generate cyclic oligoadenylates (cOA₃, cOA₄, and cOA₆) upon binding to its cognate target RNA (CTR) but not noncognate target RNA (NTR) or self RNA. The generated cOA₆ second messenger subsequently activates a nonspecific ssRNA cleavage activity in the ancillary protein L1Csm6, expressed and purified separately (FIG. 1). To harness the detectable viral RNA-stimulated DNase and RNase activities, two fluorescence reporters, an RNA oligo flanked by a fluorophore-quencher pair and a DNA oligo flanked by either the same or a different fluorophore-quencher pair were constructed (FIG. 1). The DNase and RNase activities were reconstituted by adding a model viral RNA to a reaction mixture containing L1Csm, L1Csm6, ATP, and the two fluorescence probes (FIGS. 2A-2F). FIGS. 2A-2F show one-pot reaction components for amplification detection. FIGS. 2B-2D show simultaneous rise in DNA-Alexa (shown as squares) and RNA-FAM (shown as circles) fluorescence under three different conditions tested: 10 mM MnCl₂, 0 mM ATP (FIG. 2B), 10 mM MnCl₂, 0.05 mM ATP (FIG. 2C) and 10 mM MgCl₂, 0.5 mM ATP (FIG. 2D). FIGS. 2E-2F show the augmentability of MORIARTY by combined use of 1 or 0.5 M DNA-FAM and 1 or 0.5 M RNA-FAM as probes (shown as triangles) under two different conditions tested: 10 mM MnCl₂, 0.05 mM ATP (FIG. 2E), 10 mM MgCl₂, 0.5 mM ATP (FIG. 2F). The rise in fluorescence of 1 M RNA-FAM alone are shown as circles while 1 M DNA-FAM alone are shown as squares. The optimized condition is outlined.

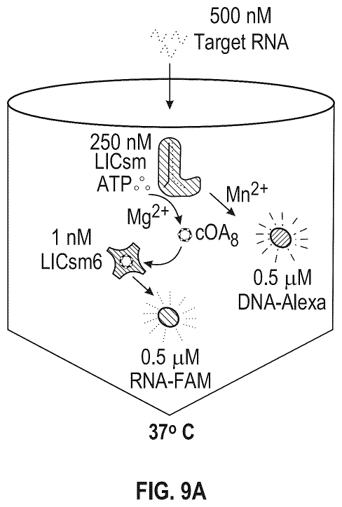

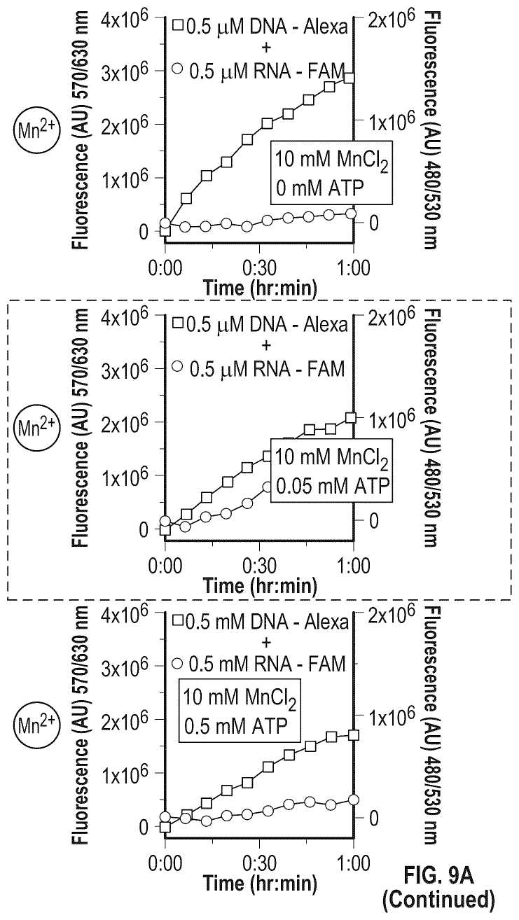

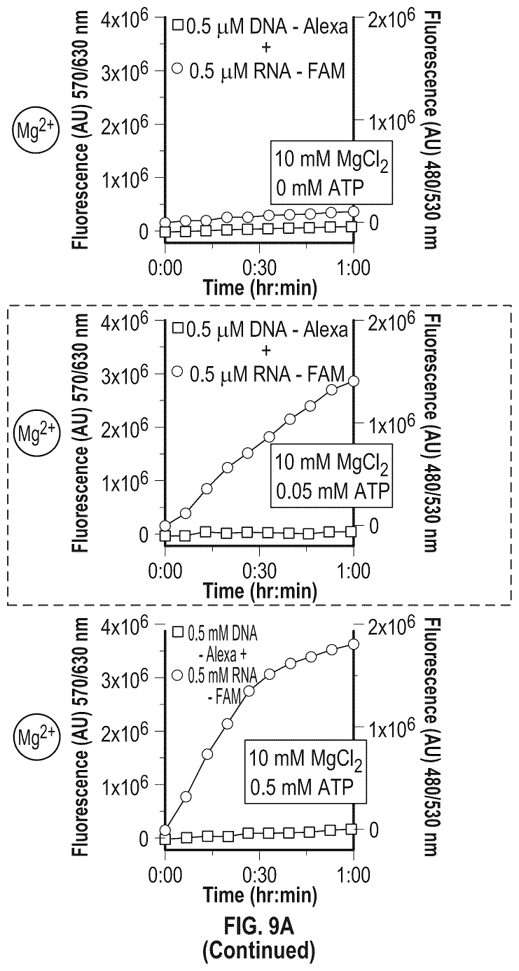

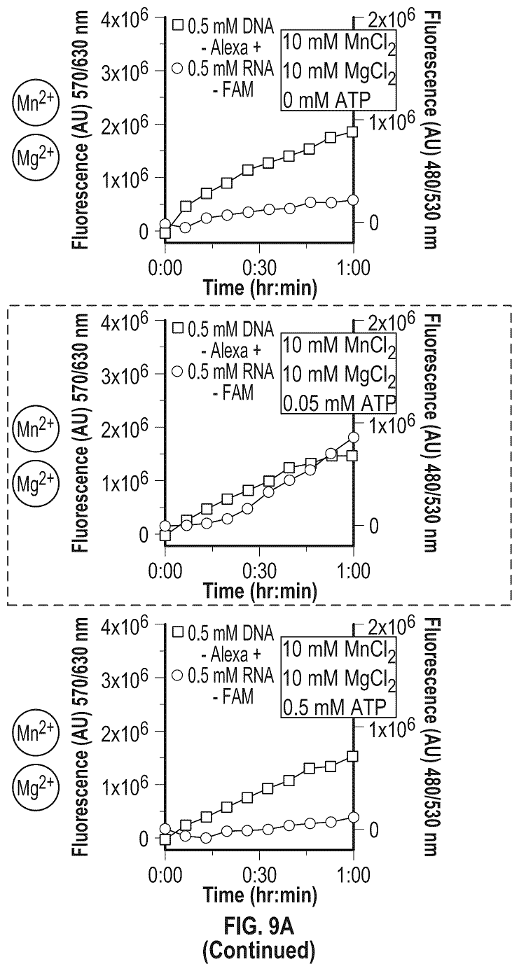

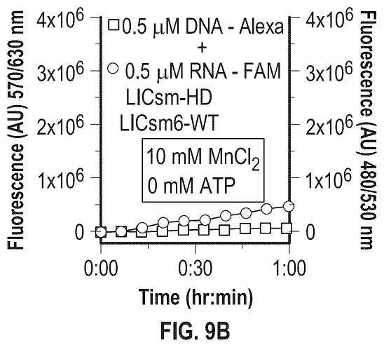

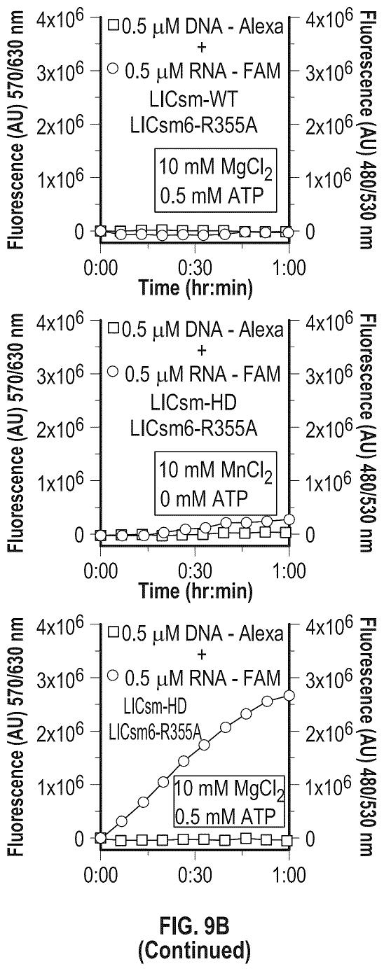

The dual functionality of L1Csm system was tracked simultaneously by using the DNA reporter flanked by Alexa594N fluorophore-quencher and the RNA reporter flanked by the Fluorescein (FAM) fluorophore-quencher with absorption/emission wavelength of 570/630 nm and 480/530 nm respectively (Table 1). Such a setup enabled detection of both activities of L1Csm1 in parallel via real-time intensity rises in two separate fluorescence channels. Consistently, mutation of the HD domain of L1Csm1 or the HEPN domain of L1Csm6 removed the Alexa595N or the FAM signal, respectively (FIG. 9A). Similarly, the absence of divalent ion Mg²⁺, thought to be essential to cOA production, abolished the FAM signal (FIGS. 2B-2F & FIG. 9B) while the absence of Mn²⁺, essential to the HD domain-mediated DNase activity, abolished the Alexa594N signal (FIGS. 2B-2F & FIG. 9B). As cOA production typically requires the presence of ATP, we found that the L1Csm6-mediated RNase activity has a strong preference for the ATP+Mg²⁺ combination while the L1Csm1-mediated DNase activity for Mn²⁺ alone. Nonetheless, strong fluorescence signals can be readily detected under either an Mn²⁺ or ATP+Mg²⁺ condition, making direct detection of viral RNA by MORIARTY possible in multiple buffer conditions.

As shown in FIGS. 9A-9B, the effects of metal ions, ATP and L1Csm/Csm6 mutations on amplification-free MORIARTY RNase (circles) and DNase (squares) probe signal, respectively. 500 nM model RNA was used as the stimulator. The RNA-FAM (circles) and DNA-Alexa594N (squares) labeled oligos were used as the fluorescence probes. The condition identified for the amplification-free MORIARTY and a T7-MORIARTY-like assays are outlined by the dashed boxes. Each mutant was tested under the condition with metal ions required for the wild-type conditions. The RNA-induced RNase activity in L1Csm6-R355A/L1Csm-HD mutants and Mg²⁺ combination was believed to be that of the GGDD domain (SEQ ID NO: 1) of Csm1.

The fluorescence signals from both RNase and DNase activities were combined with an intent to improve the overall sensitivity. To achieve this, a series of optimizations was performed to arrive at a set of Mn²⁺, Mg²⁺ and ATP combinations that gave rise to best combined FAM and Alexa594N signals (FIG. 9B). The Alexa594N fluorophore on the DNA reporter was subsequently replaced with the FAM fluorophore. It was hypothesized that a combination of DNA and RNA reporter signals could add-up under an appropriately optimized condition measured at 480/530 nm, contributing to an overall amplified fluorescence signal. Indeed, among the conditions tested, it was observed a cumulative effect of the two fluorophores in a low ATP+Mn²⁺ combined condition (FIGS. 2A-2F). Thus, MORIARTY offers augmentable and multipronged detection of viral nucleic acids that uniquely distinguishes it from other CRISPR-derived methods.

The RNA-guided Type III-A CRISPR-Cas systems can be directly activated by viral RNA transcription. It was hypothesized that a DNA template with the T7 promoter sequence and encoding the viral RNA may be used as the stimulator to MORIARTY reaction mixture supplemented with T7 transcription components, henceforth referred to as T7-MORIARTY. Under the reaction condition conducive to T7 transcription, RNA-FAM yielded strong fluorescence but not DNA-Alexa594N due to the absence of Mn²⁺ (FIGS. 3A-3F). FIGS. 3A-3F show T7 MORIARTY one-pot reaction components for transcription coupled detection. FIGS. 3B-3D show the multiprongness of T7 MORIARTY by simultaneous rise in DNA-Alexa (shown as squares) and RNA-FAM (shown as circles) fluorescence under two different conditions tested: 17 mM MgCl₂, 0.5 mM ATP, 0 mM MnCl₂ (FIGS. 3B-3C), 17 mM MgCl₂, 0.5 mM ATP, 0.5 mM MnCl₂ (FIG. 3D). The lack of T7 promoter sequence in stimulator DNA caused no rise in DNA-Alexa/RNA-FAM fluorescence (FIG. 3B). The presence of high Mg²⁺ in both the conditions supports L1Csm/L1Csm6-mediated RNA-FAM cleavage but abrogates L1Csm-mediated DNA-FAM cleavage (FIGS. 3C-3D). FIGS. 3E-3F show the augmentability of T7 MORIARTY by combined use of 1 or 0.5 M DNA-FAM and 1 or 0.5 M RNA-FAM (shown as triangles) under two different conditions tested: 17 mM MgCl₂, 0.5 mM ATP, 0 mM MnCl₂ and 1 nM L1Csm6 (FIG. 3E), 17 mM MgCl₂, 0.5 mM ATP, 0.5 mM MnCl₂ and 250 nM L1Csm6 (FIG. 3F). The rise in fluorescence of 1 M RNA-FAM alone are shown as green circles while 1 M DNA-FAM alone are shown as squares. The optimized condition is outlined.

No fluorescence was observed with the DNA stimulator lacking the T7 promotor sequence, indicating a dependence of T7-MORIARTY on transcription. Because high Mn²⁺ concentrations inhibit T7 transcription, reaction conditions were tested with varying amounts of Mn²⁺ in hopes to take advantage of both RNase and DNase signals. It was observed that supplementing T7-MORIARTY with MnCl₂ generally reduced the RNA-FAM fluorescence (FIGS. 3A-3F & FIG. 10), likely due to high-Mn²⁺-mediated inhibition of T7 transcription. FIG. 10 in particular shows the effects of metal ions and ATP on amplification-free MORIARTY with combined RNase and DNase probe signals. 500 nM model RNA was used as the stimulator. The RNA-FAM and the DNA-FAM labeled oligos were used as the fluorescence probes. The condition identified for the amplification-free MORIARTY and T7-MORIARTY assays are outlined by dashed boxes.

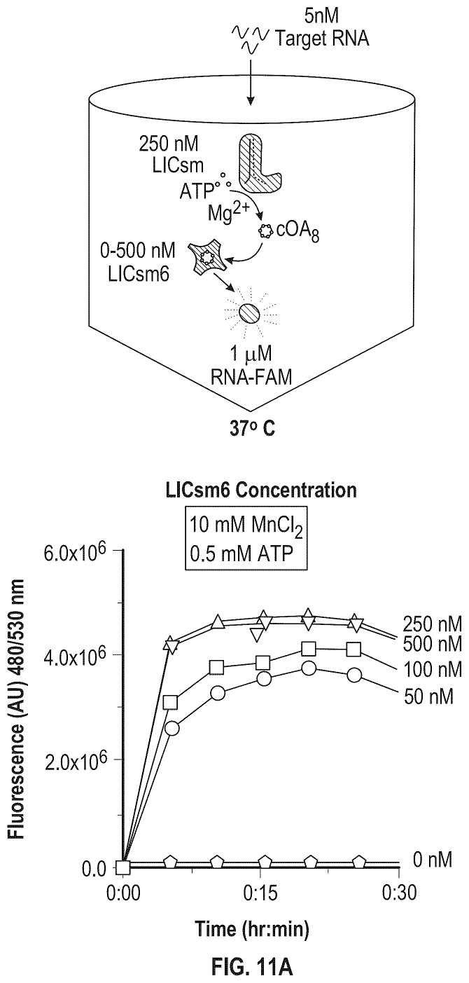

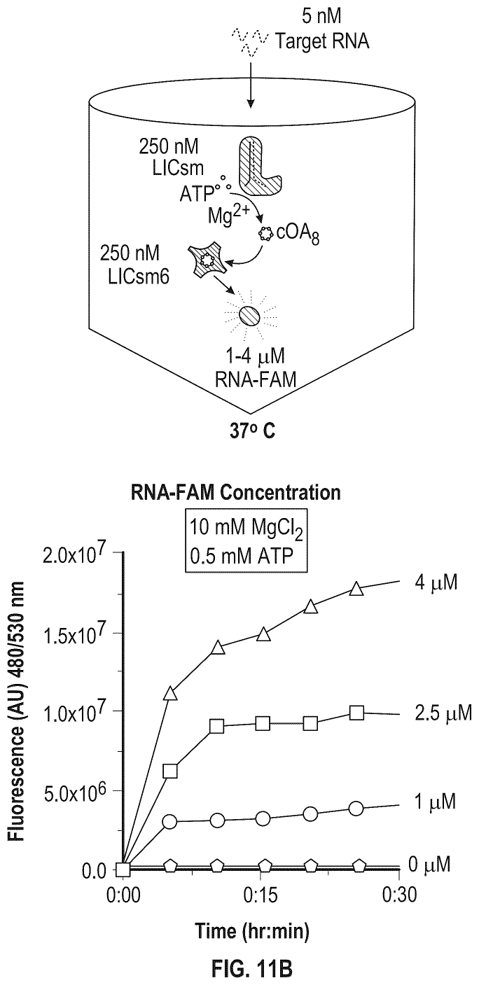

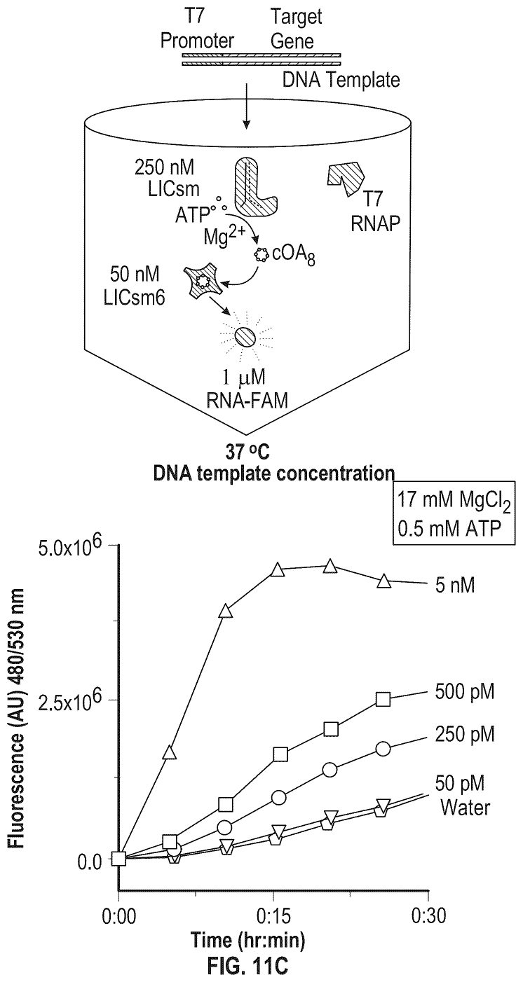

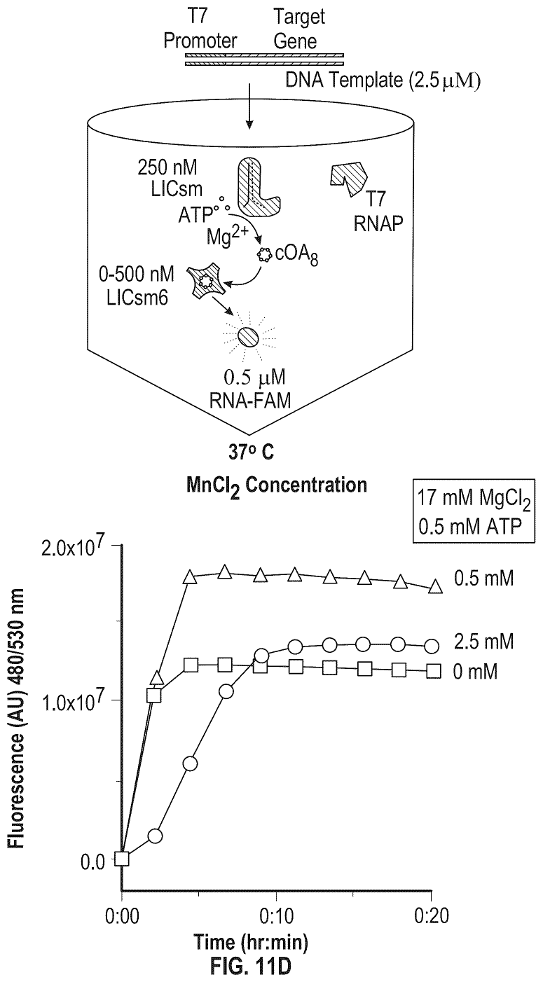

However, with a small amount of Mn² (0.5 mM) in combination with elevated Csm6, a high overall fluorescence with a non-negligible DNA reporter signal that augmented the high RNA reporter fluorescence was observed (FIGS. 3A-3F & FIG. 11A-11D).

### Example 2—Amplification-Free, Direct Detection of SARS-CoV-2 Viral RNA Via MORIARTY

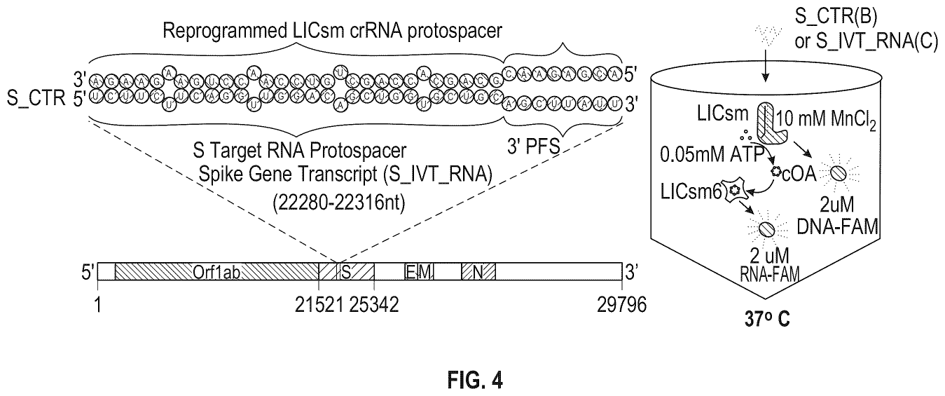

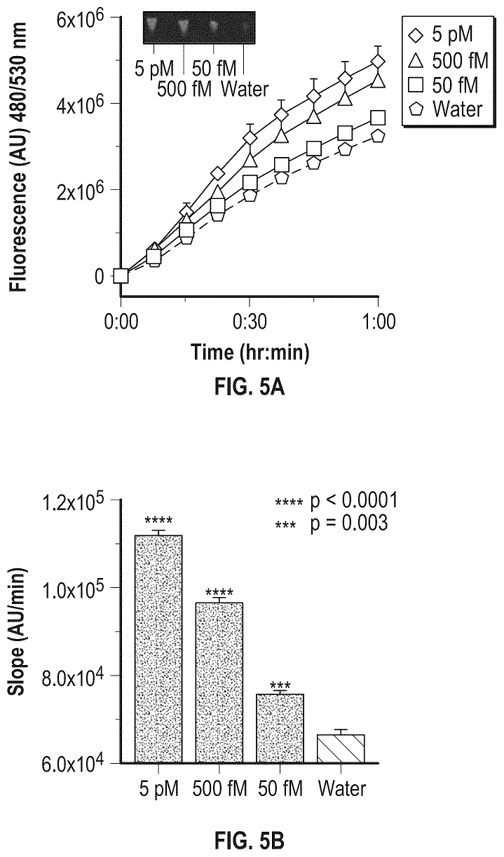

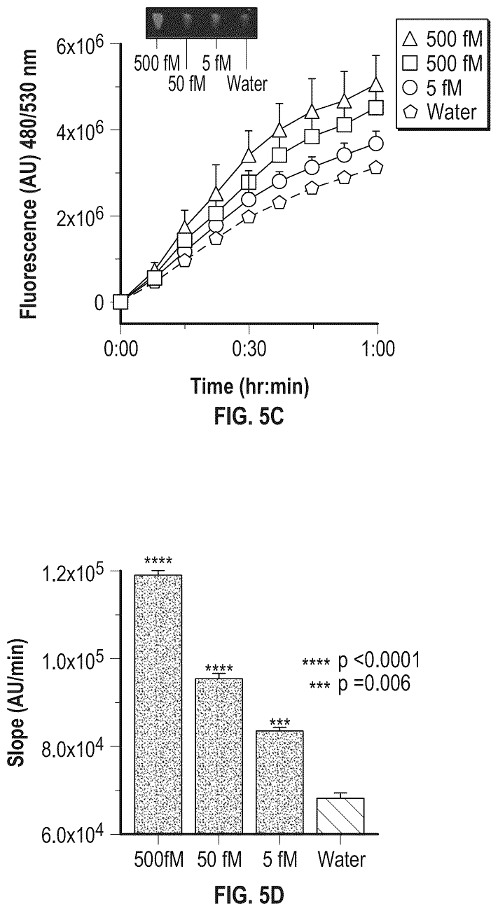

The basic principle of MORIARTY was applied in an amplification-free setting towards the detection of SARS-CoV-2 responsible for the COVID-19 pandemic by reprogramming the 29mer CRISPR RNA (crRNA) protospacer (Table 1) against the Spike (S) gene of SARS-CoV-2 (nucleotides 22280-22308, NCBI MT801051.1). The choice of the targeting region within the S gene was made such as to ensure that the 3′-protospacer flanking sequence (3′-PFS) of the viral target RNA would remain non-complementary to the 5′-handle of L1Csm crRNA (FIG. 4), ensuring activation of L1Csm upon encountering S mRNA. Expressed and copurified was the L1Csm RNP reprogrammed to detect SARS-CoV-2 S gene, hereafter referred to as L1Csm_S (Table 3). To evaluate if L1Csm_S would successfully detect its complementary CTR model RNA, hereafter referred to as S_CTR, without amplification (FIGS. 5A-5D), the L1Csm_S/L1Csm6 system was directly stimulated with S_CTR under a series of conditions (FIGS. 11A-11D). Under an optimized condition, the serially diluted 50 pM-500 fM of CTR_S could be detected with high confidence (p value<0.0001) when comparing to water (FIGS. 5A-5D). The sensitivity of L1Csm_S towards detection of in vitro transcribed S gene mRNA (S_IVS_RNA) was then evaluated. The L1Csm_S was observed to have improved sensitivity with S_IVS_RNA at 5 fM (p value=0.006) (FIGS. 5C-5D).

Specifically, as shown in FIGS. 5A-5B, the 37mer S_CTR model RNA were serially diluted and tested in MORIARTY. As a negative control, the S_CTR sample was replaced by water. The rise in fluorescence corresponding to 5 pM, 500 fM and 50 fM are shown relative to water. As shown in FIGS. 5C-5D, the S_IVT_RNA samples were serially diluted and tested in MORIARTY. As a negative control, the S_IVT_RNA sample was replaced by water. The rise in fluorescence corresponding to 500 fM, 50 fM and 5 fM are shown relative to water. The reaction products imaged against Fluorescein wavelength with BioImager are shown. The slope of the curves (right) was determined by employing simple linear regression to the data obtained from the initial 30 minutes of the experiment performed in triplicates. All the slopes were compared to the negative control by performing Ordinary one-way analysis of variance (ANOVA) and Dunnett's multiple comparisons test. ****=p<0.0001, ***=p<0.001 and ns=non-significant.

As shown in FIGS. 11A-11D, the effects of concentrations of L1Csm6 (FIG. 11A), probe (FIG. 11B), DNA template (FIG. 11C) and supplementary Mn²⁺ (FIG. 11D) on T7-MARIARTY. Only RNA-FAM was included as the fluorescence probe. A range of L1Csm6 concentrations were used in a Mg²⁺/ATP condition (FIG. 11A). Note concentration greater than 250 nM did not yield better signal. A range of RNA-FAM concentration was tested in the Mg²⁺/ATP condition (FIG. 11B). Increasing the probe concentration increased the signal. Synthetic DNA oligos containing T7 promoter sequence at various concentrations were used as stimulator to T7-MORIARTY (FIG. 11C). The effect of supplementing a small amount of Mn2+ ions on T7-MORIARTY with optimized L1Csm6 (FIG. 11D).

### Example 3—Attomolar Detection of SARS-CoV-2 Virus with Amplification-Coupled MORIARTY

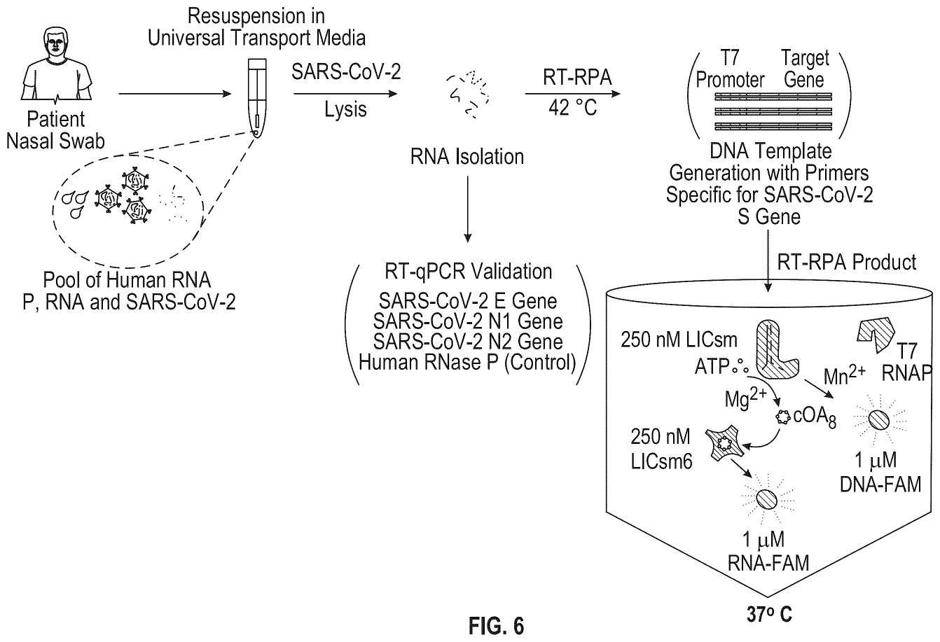

Whether the sensitivity of MORIARTY could be improved when it was coupled with a pre-amplification step with reverse transcription and recombinase-polymerase amplification (RT-RPA) was examined. In this application, viral RNA was first reverse transcribed by a reverse transcriptase (RT) followed by dsDNA synthesis from a T7-promoter containing primer and a downstream primer via three recombinase-polymerase amplification (RPA) enzymes: a recombinase, single-stranded DNA-binding protein (SSB) and strand-displacing polymerase (FIG. 6). The RT-RPA protocols optimized for the detection of the S-gene of SARS-CoV-2 were used to test the sensitivity of MORIARTY to RT-RPA reaction products from varying amount of viral RNA.

MORIARTY can be extremely sensitive to the RT-RPA products amplified from the in vitro transcribed S gene mRNA, hereafter referred to as S_IVT_RNA. A 25 μL RT-RPA reaction was performed using 2.5 μL S_IVT_RNA at varying concentrations by incubating the reaction mix at a 42° C. water bath for 25 minutes. Addition of 15 μL of RT-RPA product in a 100 μL optimized T7 MORIARTY reaction resulted in a clear rise in total fluorescence above water as low as 200 aM, or roughly 100 copies/mL by a qPCR procedure.

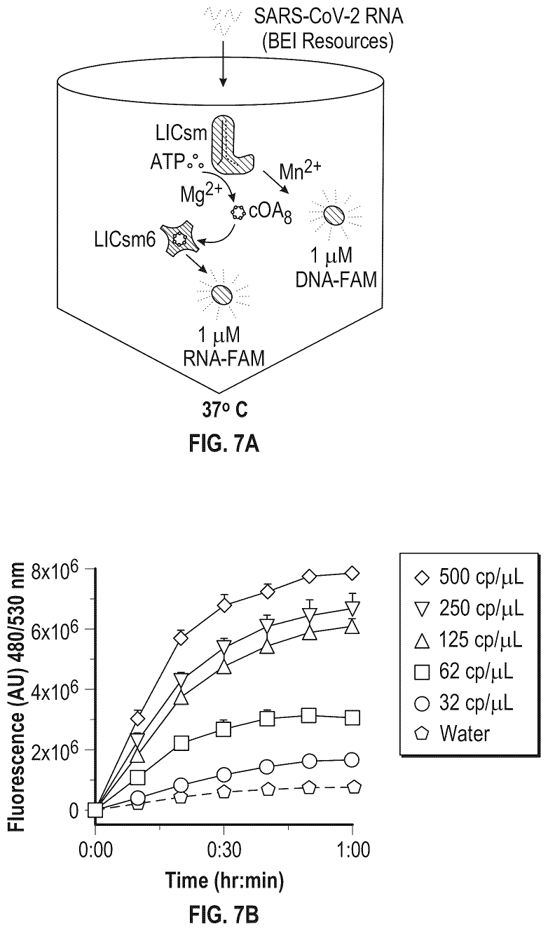

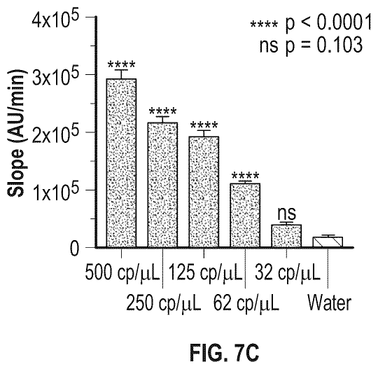

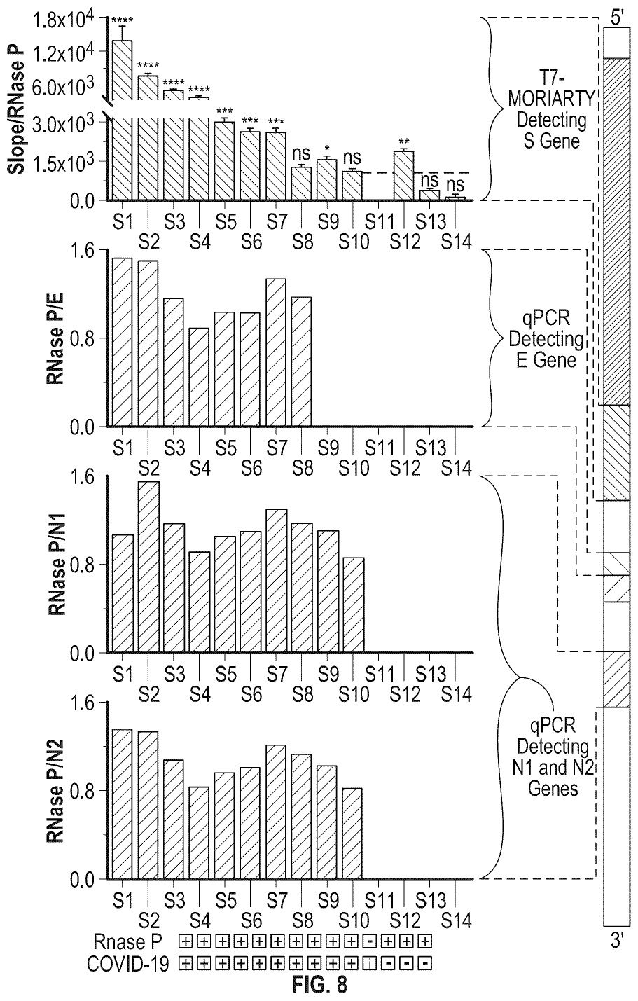

MORIARTY was next applied to the detection of Quantitative PCR (qPCR) control RNA extracted from heat-inactivated SARS-CoV-2 virus (BEI NR-52347) with known viral titters (50,000 cp/mL). Serial dilutions were performed from the stock in a range of 500-31.25 cp/μl in water and used them in RT-RPA and the subsequent T7 MORIARTY reactions. Samples with copy numbers higher than 62.5 cp/μL produced statistically significant rise above water in multi-replicate experiments (FIGS. 7A-7C). As shown in FIGS. 7A-7C, the SARS-CoV-2 control RNA samples (BEI NR-52347) were serially diluted, added to RT-RPA reaction and tested in T7 MORIARTY. As a negative control, RT-RPA treated water was used instead of RT-RPA treated sample. The rise in fluorescence corresponding to 500 cp/μL, 250 cp/μL, 125 cp/μL, 62.5 cp/μL and 31.25 cp/μL relative to water are shown. The slope of the curves was determined by employing simple linear regression to the data obtained from the initial 30 minutes of the experiment performed in triplicates. All the slopes were compared to the negative control by performing Ordinary one-way analysis of variance (ANOVA) and Dunnett's multiple comparisons test. ****=p<0.0001, ***=p<0.001 and ns=non-significant.

Finally, MORIARTY was applied to human patient samples obtained from a COVID-19 testing center. A total of fourteen patient samples were obtained and subjected to RNA extraction (QIAamp viral mini kit). The extracted RNA from each patient was split for simultaneous detection with MORIARTY and an FDA approved qPCR procedure, respectively. Since the qPCR method detected four targets, human RNase P, SARS-CoV-2 N1, N2 and E genes, while the MORIARTY of this example detected S gene in each sample, the detected viral signals were normalized by using the counts of human RNase P for each patient. For qPCR results, the quotients of human RNase P count and each of the three viral count numbers computed and plotted, respectively. For MORIARTY results, the quotient of fluorescence slope and human RNase P count for each patient was computed and plotted. For both results, high quotient values were indicative of high viral titter. Thus, a set of consistent results were obtained from two different experiments for comparison (FIG. 8).

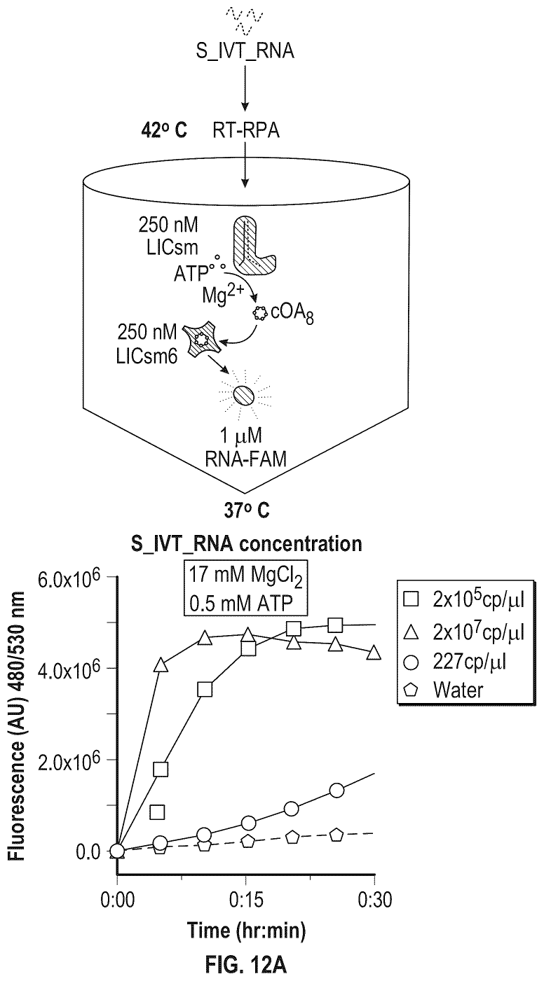

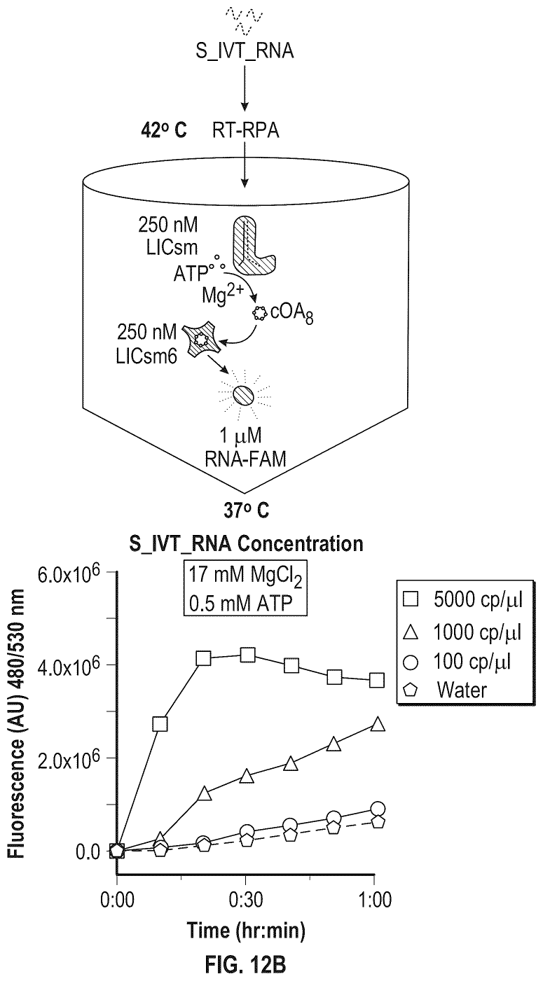

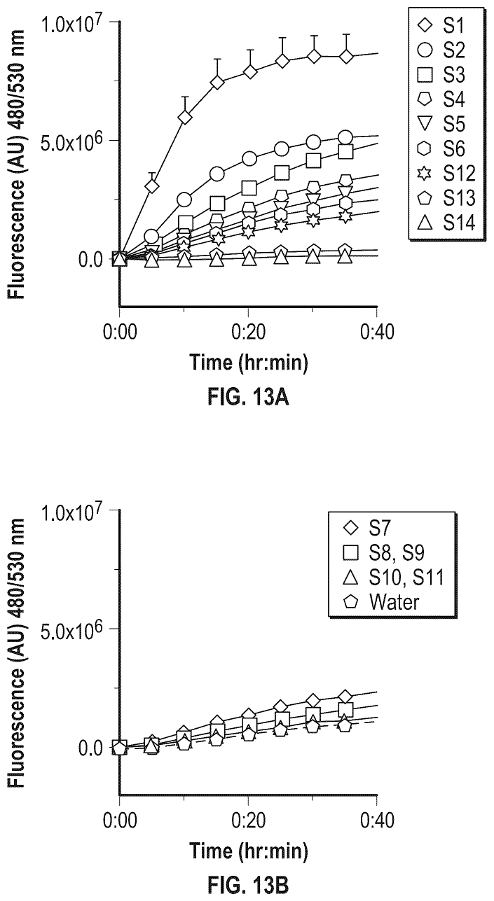

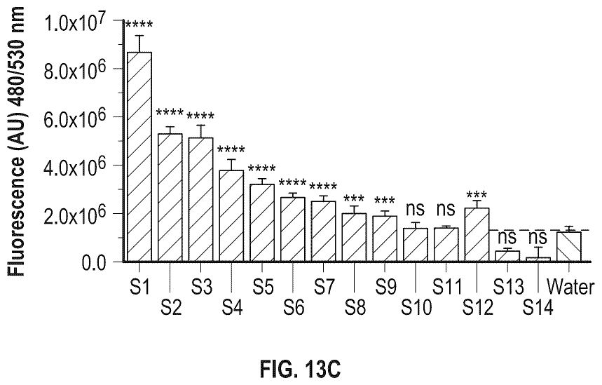

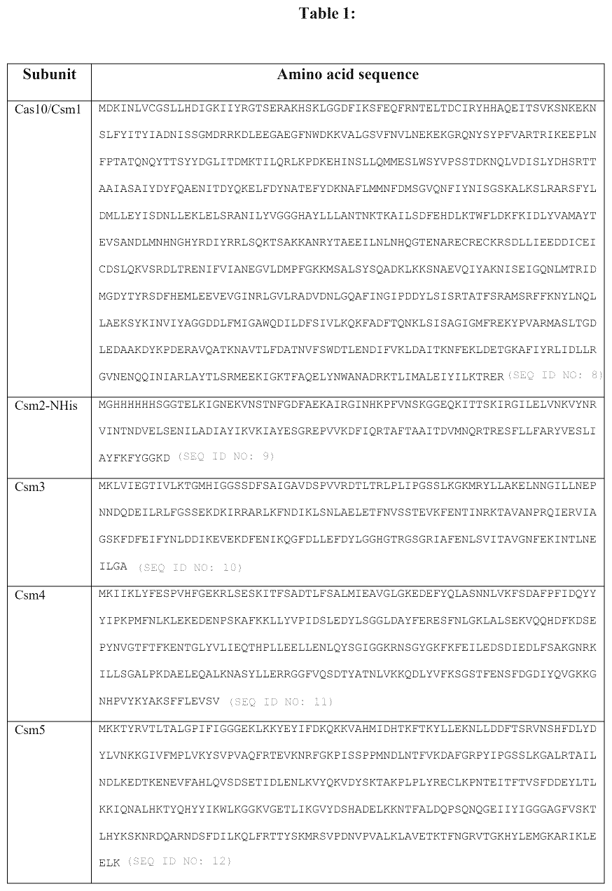

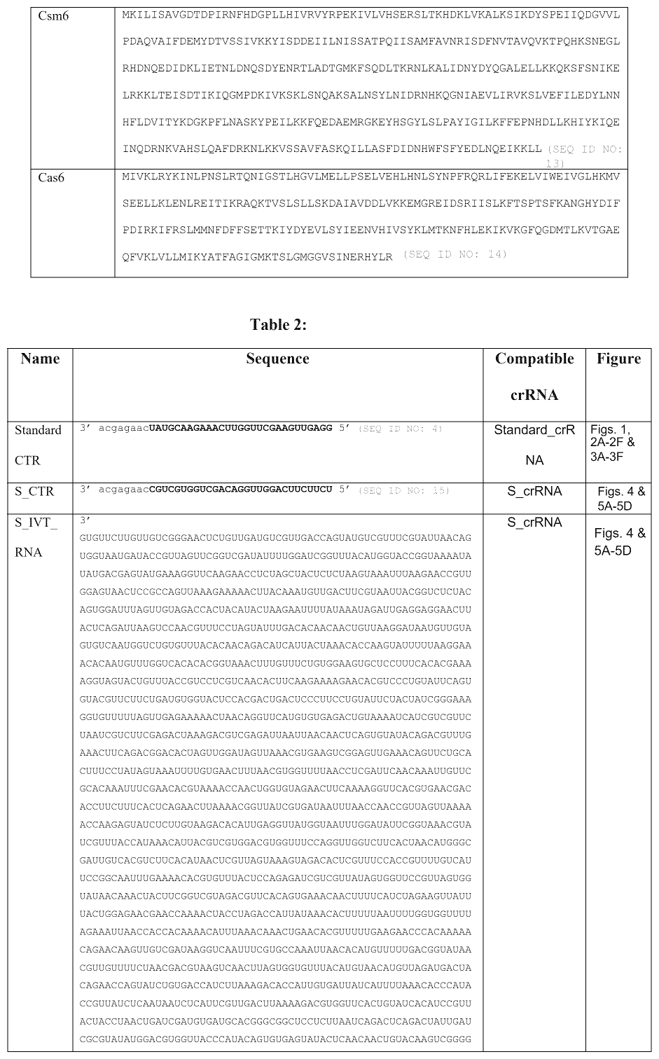

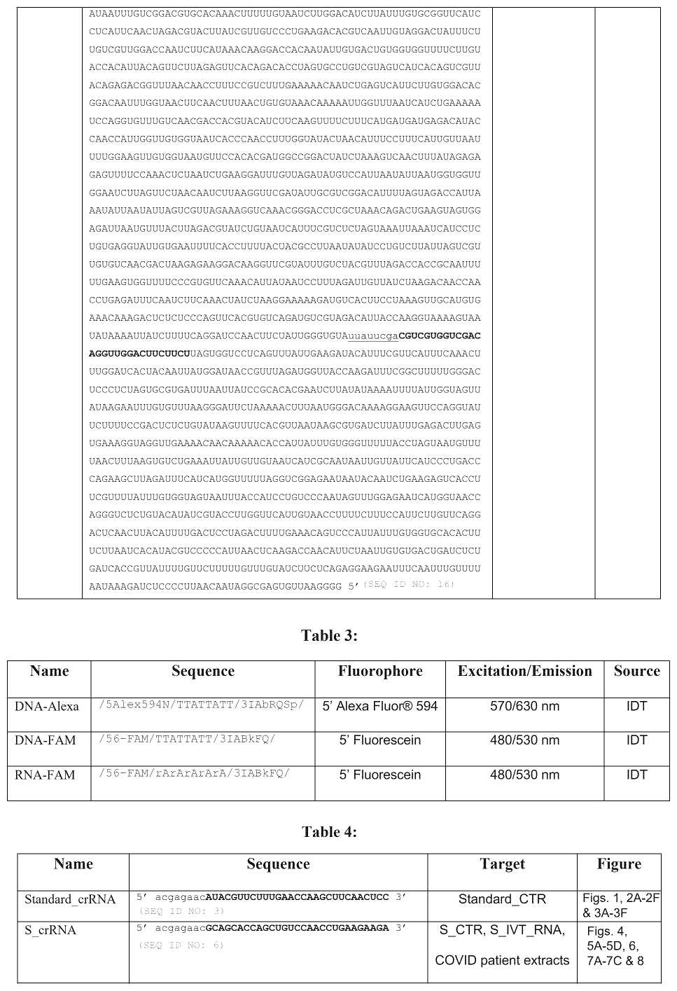

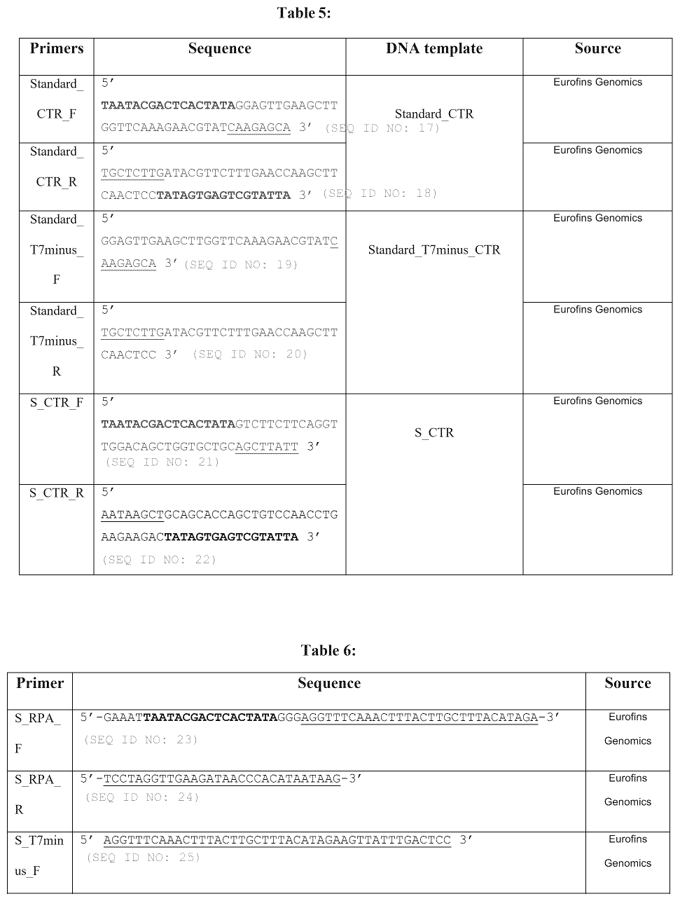

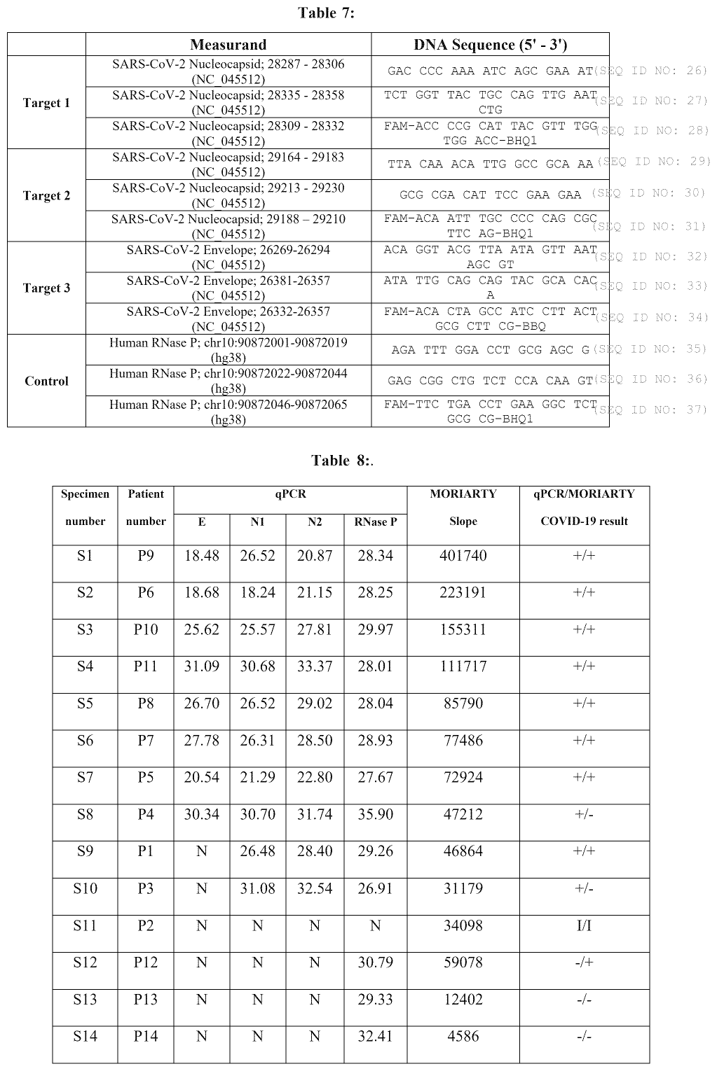

As shown in FIG. 8, the viral RNA samples extracted from 14 patients were RT-RPA treated and tested in T7-MORIARTY. T7-MORIARTY results were obtained as in FIGS. 7B-7C, subtracted from that of water, normalized with individual RNase P counts and depicted as a bar diagram with statistical significance values. The qPCR results were plotted as bar diagrams for the three targets (E, N1, and N2) as quotients of Cₜ value for RNase P and that of each target. In general, a good agreement between MORIARTY results with the detected E gene titer with qPCR was observed (FIG. 8). FIG. 12A shows in vitro transcribed S-gene mRNA (S_IVT_RNA) at various concentrations for RT-RPA-T7-MORIARTY. The concentration of the S_IVT_RNA was determined by q-RT-PCR based on model SARS-CoV-2 virus (BEI NR-52347). In FIG. 12B, model SARS-CoV-2 virus with known concentrations (BEI NR-52347) were used to test the requirement for T7 promoter sequence in the S-gene specific primers. T7-minus primer denotes the forward primer specific for S-gene but without T7 promoter sequence. Additionally, the raw data of rise in fluorescence of patient samples was tested in RT-RPA-T7-MORIARTY (FIGS. 13A-13B) and a bar diagram of the fluorescence intensities at 40 min time point (FIG. 13C) of the 14 patient samples was prepared. Each sample was tested in triplicate as two groups of experiments along with either a negative sample or water. The grey dotted line represents intensity measured on RT-RPA treated water. Intensities were compared to the negative control by performing Ordinary one-way analysis of variance (ANOVA) and Dunnett's multiple comparisons test. ****=p<0.0001, ***=0.003<p<0.006 and ns=non-significant.

MORIARTY correctly diagnosed three of the four qPCR-diagnosed negative and seven of the eight qPCR-diagnosed positive patients with excellent statistics (FIG. 8). Interestingly, the qPCR results for the N1 and N2 genes agreed with those of MORIARTY to a less degree, likely reflecting the difference in target site accessibility or annealing thermodynamics between the N and the S gene primers.

### Material and Methods

### Cloning

The pACYC Lactococcus lactis Csm (L1Csm) effector module plasmid encoding Cas6, Csm1-6 and CRISPR locus was as described previously. The standard L1Csm effector complex was reprogrammed to detect the S gene of SARS-CoV-2 by replacing the 29mer protospacer region with 29mer gene-complementary sequence via Q5 mutagenesis. The desired mutations including Csm1 H13A, Csm1 D14N, Csm3 D30A and Csm6 R355A were also introduced by Q5 mutagenesis. For multiplexing, three new clones of L1Csm_S were designed: L1Csm_S7 (nucleotides 24702-24730, MT801051.1), L1Csm_S8 (nucleotides 25061-25089, MT801051.1) and L1Csm_S9 (nucleotides 25092-25120, MT801051.1) each of which harbored Csm1 H13A and Csm3 D30A mutations. The reprogramming of crRNA protospacer region was achieved as described previously. Briefly, two BbsI sites were introduced at the 3′-end of the repeat and the 5′-end of the HDV ribozyme region, upstream and downstream of the target protospacer region in the L1Csm plasmid using Q5 mutagenesis. The modified plasmid was restriction digested using BbsI enzyme at 37° C. for 60 min and the digested product was gel extracted, purified using a gel extraction kit. To create individual L1Csm multiplexing clones: S7, S8 and S9, the linearized plasmid was incubated with the respective 29mer oligos at 37° C. for 10 min for ligation using DNA ligase. The ligated DNA was transformed into the DH5a competent cells, plated on LB-agar plate and incubated at 37° C. for 16 hours. Next, two colonies from each plate were picked, grown overnight and plasmid DNA was isolated using a kit. All the clones were verified using sequencing primers.

### Protein Expression and Purification

The standard and reprogrammed L1Csm complexes were all expressed and purified as described previously. Briefly, L1Csm complexes encoded in all-in-one pACYC plasmid (FIG. 1) were produced in Escherichia coli NICO stain using 0.3 mM isopropyl 3-D-1 thiogalactopyranoside (IPTG) for induction of protein expression. The L1Csm2 subunit harbored N-terminal His₆-tag (SEQ ID NO: 2) that enabled isolation of L1Csm ribonucleoprotein complex using Ni-immobilized affinity chromatography. The complexes were further purified using size-exclusion chromatography and stored in 20 mM HEPES pH 7.5, 200 mM NaCl, 5 mM MgCl₂, 14 mM 2-mercaptoethanol. As L1Csm6 is not a component of the effector complex, it was produced separately using pLlCsm6 plasmid (FIG. 1) as described previously and stored in 20 mM HEPES pH 7.5, 300 mM NaCl, 14 mM 2-mercaptoethanol. The T7 RNA polymerase was produced as described previously.

In Vitro Transcription of S Gene mRNA

The bacterial expression plasmid for SARS-CoV-2 surface glycoprotein (Spike protein) pGBW-m4046887 (Addgene) was cultured in LB media supplemented with 35 g/ml Chloramphenicol at 37° C. overnight and subjected to plasmid DNA extraction kit. The extracted plasmid DNA was subjected to BamH1 restriction digestion to linearize the plasmid, run on an agarose gel and the linearized DNA was gel-extracted. The gel-extracted DNA was used as starting material with suitable primers to generate a dsDNA template for in vitro transcription. The S_IVT_RNA was transcribed using 1 g template DNA in a reaction containing 1× transcription buffer (10× consists of 500 mM Tris pH 8.0, 100 mM DTT 200 mM MgCl₂), 5 mM rNTPs, 480 g/mL T7 RNA polymerase. The transcription reaction was incubated at 37° C. for 3 hrs. The RNA transcript was purified using Monarch RNA Cleanup kit, eluted in water, aliquoted, flash frozen using liquid nitrogen and stored at −80° C.

### RT-RPA

The RT-RPA was performed using a kit as previously described. For each lyophilized pellet provided by the kit, a 50 μL reaction mix containing 29.5 μL of rehydration buffer, 0.5 M each of forward and reverse primers and 100 U Protoscript reverse transcriptase was prepared. The master-mix was added to the RPA tube on ice to resuspend the pellet. After the pellet was dissolved completely, 5 μL RNA samples extracted from patient nasopharyngeal swab using QIAamp viral mini kit was added. To initiate the RT-RPA reaction, 14 mM Magnesium Acetate (provided with the kit) was added and the reactions were incubated for 30 minutes at 42° C. with intermittent mixing every 10 minutes. After the reaction was complete, the RT-RPA product was transferred to ice until T7-MORIARTY detection assay was set up.

### Amplification-Free MORIARTY

The overall design of Amplification-free MORIARTY methodology of the foregoing examples was optimized from the previously reported fluorescent reporter assay. To depict multiprongness of MORIARTY, DNA-probe and RNA-probe harbored 5′-Alexa Fluor594 and 5′ 6-FAM fluorescent dyes respectively. The reactions were performed in 1× TAPA buffer (10× consists of 330 mM Tris acetate pH 7.6 at 32° C., 660 mM Potassium acetate) containing 0.5 pM DNA-Alexa, 0.5 M RNA-FAM (IDT), 250 nM L1Csm effector complex, 1 nM L1Csm6, 10 mM MgCl₂/MnCl₂, 0-0.5 mM ATP and 0-500 nM target RNA at 37° C. The dual fluorescence was simultaneously measured on Spectramax ID5 multi-mode microplate reader (Molecular Devices) using 480 nm/530 nm (to track cleavage of RNA-FAM) and 570 nm/630 nm (to track cleavage of DNA-Alexa) excitation/emission wavelength at 1 min intervals. The reactions were performed in triplicates and averaged for the final plots. To depict the augmentability of MORIARTY, the reactions were performed similarly, but with DNA-Alexa replaced by DNA-FAM. The fluorescence was measured using 480 nm/530 nm excitation/emission wavelength at 1 min intervals. The reaction products were transferred to 0.2 ml PCR tubes and imaged against Fluorescein wavelength filter for direct visualization.

### T7-MORIARTY

The T7-MORIARTY methodology of the foregoing examples was designed to track L1Csm co-transcriptional activation of template viral DNA obtained from RT-RPA step. All T7-MORIARTY reactions were done using RNA-FAM and DNA-FAM fluorescent probes at a reaction volume of 25 μL or 100 μL. The reactions were performed in a buffer cocktail of 1× TAPA buffer (10× consists of 330 mM Tris acetate pH 7.6 at 32° C., 660 mM Potassium acetate) and 1× HEPES transcription buffer (10× contains 300 mM K-HEPES pH 7.6, 20 mM Spermidine, 0.1% Triton X-100, 170 mM MgCl₂) containing 1 M DNA-FAM (IDT), 1 M RNA-FAM (IDT), 250 nM L1Csm effector complex, 250 nM L1Csm6, 10 mM MgCl₂, 0.5 mM MnCl₂, 0.5 mM rNTPs, 10 mM TCEP, 60 g/mL T7 RNA polymerase and 0-500 nM target RNA at 37° C.

The fluorescence was measured on Spectramax ID5 multi-mode microplate reader using 480 nm/530 nm excitation/emission wavelength at 5 min intervals. The reactions were performed in triplicates and averaged for the final plots. The reaction products were transferred to 0.2 mL PCR tubes and imaged against Fluorescein wavelength.

Target RNA Sequences and crRNA Sequences

Target RNA sequences are provided in Table 2. The 37mer target sequences are shown highlighting 29mer region complementary to crRNA protospacer (bold). The corresponding 8-nt 3′-protospacer flanking sequence (3′-PFS) is shown in small letters. The 3822 b long S_IVT_RNA sequence is shown highlighting the 3′-PFS (small letters/underline) and target RNA PFS (in highlight/bold). Table 4 provides crRNA sequences. The 37mer crRNA sequences are shown highlighting reprogrammable 29mer protospacer region (bold). The 8-nt crRNA 5′-handle sequence (small letters) originates from the repeat sequence processed by Cas6.

### DNA Templates

Table 5 provides a compilation of DNA templates used in FIG. 1, FIGS. 2A-2F, and FIGS. 3A-3F. The double stranded DNA templates for T7-MORIARTY were prepared by annealing non-coding and coding strands ordered as single-stranded forward and reverse primers. The T7 promoter region in the forward primer and its complementary sequence in the reverse primer are shown in bold. The region corresponding to 8-nt 3′-PFS of transcript is underlined.

### RPA Primers

Table 6 provides a compilation of RNA primers used in FIG. 6, FIGS. 7A-7C, and FIG. 8. The RPA primers used in RT-RPA-T7-MORIARTY were designed to detect S gene. The T7 promoter region is highlighted in bold. The underlined region represents primer segment annealing to the target

### Target DNA Sequences

Table 7 provides a compilation of the target DNA sequences of Nucleocapsid (N), Envelope (E) genes of SARS-CoV-2 and human RNase P control used in qPCR validation, used in FIG. 6, FIGS. 7A-7C, and FIG. 8.

Raw qPCR and T7-MORIARTY Results

Table 8 provides raw qPCR and T7-MORIARTY results of 14 patient samples tested in FIG. 6, FIGS. 7A-7C, and FIG. 8. For each patient, Cₜ values corresponding to SARS-CoV-2 E, N1 and N2 genes, human RNase P and MORIARTY slope values are shown. “+” represents positive diagnosis, “−” represents negative diagnosis and “I” represents invalidated sample due to undetectable human RNase P.

That which is claimed is:

1. A system for detecting a virus comprising:
a L1Csm effector complex;
a L1Csm6 protein;
a first reporter comprising an RNA oligo flanked by a first fluorophore-quencher pair; and
a second reporter comprising a DNA oligo flanked by a second fluorophore-quencher pair.

2. The system of claim 1, further comprising a buffer.

3. The system of claim 2, wherein the buffer comprises Mg2+, ATP, or a combination thereof.

4. The system of claim 1, further comprising a T7 promoter sequence.

5. The system of claim 1, further comprising a sample comprising a virus to be detected, wherein the system can detect the virus at 5 fM or less.

6. The system of claim 1, wherein the first reporter comprises an RNA oligo flanked by a first fluorophore-quencher pair, and the second reporter comprises a DNA oligo flanked by a second fluorophore-quencher pair.

7. The system of claim 1, wherein the first fluorophore-quencher pair and the second fluorophore-quencher pair are the same.

8. The system of claim 1, wherein the virus comprises a spike protein, and the spike protein comprises the cognate target RNA.

9. The system of claim 1, wherein the virus is SARS-CoV-2.

10. The system of claim 1, wherein the system is capable of detecting both the detectable DNase activity and the detectable RNase activity in the virus.

11. The system of claim 1, wherein the virus is not amplified.

12. The system of claim 1, wherein the effector complex and the L1Csm6 protein are contained in a liquid.

13. The system of claim 12, wherein a concentration of the effector complex in the liquid is about 200 nM to about 300 nM, and a concentration of the L1Csm6 protein is about 0.1 nM to about 3 nM.

14. The system of claim 12, wherein the liquid comprises a buffer.

15. The system of claim 1, wherein the system contacts a sample comprising the virus, wherein the virus comprises a cognate target RNA, wherein the effector complex binds to the cognate target RNA, and the binding of the effector complex to the cognate target RNA produces at least one messenger comprising a cyclic oligoadenylate.

16. The system of claim 15, wherein the at least one messenger comprises cOA₆.

17. The system of claim 15, wherein the at least one messenger activates nonspecific ssRNA cleavage activity in the ancillary protein to produce detectable DNase activity, detectable RNase activity, or a combination thereof.


SEQUENCE LISTING (37 sequences)

SEQ ID NO: 1 - protein, 4 aa, unidentified
  REGION 1..4: note = Description of Unknown: GGDD motif sequence
GGDD

SEQ ID NO: 2 - protein, 6 aa, synthetic construct
  REGION 1..6: note = Description of Artificial Sequence: Synthetic 6xHis tag
HHHHHH

SEQ ID NO: 3 - RNA, 37 nt, synthetic construct
  misc_feature 1..37: note = Description of Artificial Sequence: Synthetic oligonucleotide
  source 1..37: mol_type = other RNA
acgagaacatacgttctttgaaccaagcttcaactcc

SEQ ID NO: 4 - RNA, 37 nt, synthetic construct
  misc_feature 1..37: note = Description of Artificial Sequence: Synthetic oligonucleotide
  source 1..37: mol_type = other RNA
ggagttgaagcttggttcaaagaacgtatcaagagca

SEQ ID NO: 5 - RNA, 37 nt, synthetic construct
  misc_feature 1..37: note = Description of Artificial Sequence: Synthetic oligonucleotide
  source 1..37: mol_type = other RNA
ggagttgaagcttggttcaaagaacgtatgttctcgt

SEQ ID NO: 6 - RNA, 37 nt, synthetic construct
  misc_feature 1..37: note = Description of Artificial Sequence: Synthetic oligonucleotide
  source 1..37: mol_type = other RNA
acgagaacgcagcaccagctgtccaacctgaagaaga

SEQ ID NO: 7 - RNA, 37 nt, synthetic construct
  misc_feature 1..37: note = Description of Artificial Sequence: Synthetic oligonucleotide
  source 1..37: mol_type = other RNA
tcttcttcaggttggacagctggtgctgcagcttatt

SEQ ID NO: 8 - protein, 757 aa, synthetic construct
  REGION 1..757: note = Description of Artificial Sequence: Synthetic polypeptide
MDKINLVCGSLLHDIGKIIYRGTSERAKHSKLGGDFIKSFEQFRNTELTDCIRYHHAQEITSVKSNKEKNSLFYITYIADNISSGMDRRKDLEEGAEGFNWDKKVALGSVFNVLNEKEKGRQNYSYPFVARTRIKEEPLNFPTATQNQYTTSYYDGLITDMKTILQRLKPDKEHINSLLQMMESLWSYVPSSTDKNQLVDISLYDHSRTTAAIASAIYDYFQAENITDYQKELFDYNATEFYDKNAFLMMNFDMSGVQNFIYNISGSKALKSLRARSFYLDMLLEYISDNLLEKLELSRANILYVGGGHAYLLLANTNKTKAILSDFEHDLKTWFLDKFKIDLYVAMAYTEVSANDLMNHNGHYRDIYRRLSQKTSAKKANRYTAEEILNLNHQGTENARECRECKRSDLLIEEDDICEICDSLQKVSRDLTRENIFVIANEGVLDMPFGKKMSALSYSQADKLKKSNAEVQIYAKNISEIGQNLMTRIDMGDYTYRSDFHEMLEEVEVGINRLGVLRADVDNLGQAFINGIPDDYLSISRTATFSRAMSRFFKNYLNQLLAEKSYKINVIYAGGDDLFMIGAWQDILDFSIVLKQKFADFTQNKLSISAGIGMFREKYPVARMASLTGDLEDAAKDYKPDERAVQATKNAVTLFDATNVFSWDTLENDIFVKLDAITKNFEKLDETGKAFIYRLIDLLRGVNENQQINIARLAYTLSRMEEKIGKTFAQELYNWANADRKTLIMALEIYILKTRER

SEQ ID NO: 9 - protein, 150 aa, synthetic construct
  REGION 1..150: note = Description of Artificial Sequence: Synthetic polypeptide
MGHHHHHHSGGTELKIGNEKVNSTNFGDFAEKAIRGINHKPFVNSKGGEQKITTSKIRGILELVNKVYNRVINTNDVELSENILADIAYIKVKIAYESGREPVVKDFIQRTAFTAAITDVMNQRTRESFLLFARYVESLIAYFKFYGGKD

SEQ ID NO: 10 - protein, 214 aa, synthetic construct
  REGION 1..214: note = Description of Artificial Sequence: Synthetic polypeptide
MKLVIEGTIVLKTGMHIGGSSDFSAIGAVDSPVVRDTLTRLPLIPGSSLKGKMRYLLAKELNNGILLNEPNNDQDEILRLFGSSEKDKIRRARLKFNDIKLSNLAELETFNVSSTEVKFENTINRKTAVANPRQIERVIAGSKFDFEIFYNLDDIKEVEKDFENIKQGFDLLEFDYLGGHGTRGSGRIAFENLSVITAVGNFEKINTLNEILGA

SEQ ID NO: 11 - protein, 297 aa, synthetic construct
  REGION 1..297: note = Description of Artificial Sequence: Synthetic polypeptide
MKIIKLYFESPVHFGEKRLSESKITFSADTLFSALMIEAVGLGKEDEFYQLASNNLVKFSDAFPFIDQYYYIPKPMFNLKLEKEDENPSKAFKKLLYVPIDSLEDYLSGGLDAYFERESFNLGKLALSEKVQQHDFKDSEPYNVGTFTFKENTGLYVLIEQTHPLLEELLENLQYSGIGGKRNSGYGKFKFEILEDSDIEDLFSAKGNRKILLSGALPKDAELEQALKNASYLLERRGGFVQSDTYATNLVKKQDLYVFKSGSTFENSFDGDIYQVGKKGNHPVYKYAKSFFLEVSV

SEQ ID NO: 12 - protein, 353 aa, synthetic construct
  REGION 1..353: note = Description of Artificial Sequence: Synthetic polypeptide
MKKTYRVTLTALGPIFIGGGEKLKKYEYIFDKQKKVAHMIDHTKFTKYLLEKNLLDDFTSRVNSHFDLYDYLVNKKGIVFMPLVKYSVPVAQFRTEVKNRFGKPISSPPMNDLNTFVKDAFGRPYIPGSSLKGALRTAILNDLKEDTKENEVFAHLQVSDSETIDLENLKVYQKVDYSKTAKPLPLYRECLKPNTEITFTVSFDDEYLTLKKIQNALHKTYQHYYIKWLKGGKVGETLIKGVYDSHADELKKNTFALDQPSQNQGEIIYIGGGAGFVSKTLHYKSKNRDQARNDSFDILKQLFRTTYSKMRSVPDNVPVALKLAVETKTFNGRVTGKHYLEMGKARIKLEELK

SEQ ID NO: 13 - protein, 409 aa, synthetic construct
  REGION 1..409: note = Description of Artificial Sequence: Synthetic polypeptide
MKILISAVGDTDPIRNFHDGPLLHIVRVYRPEKIVLVHSERSLTKHDKLVKALKSIKDYSPEIIQDGVVLPDAQVAIFDEMYDTVSSIVKKYISDDEIILNISSATPQIISAMFAVNRISDFNVTAVQVKTPQHKSNEGLRHDNQEDIDKLIETNLDNQSDYENRTLADTGMKFSQDLTKRNLKALIDNYDYQGALELLKKQKSFSNIKELRKKLTEISDTIKIQGMPDKIVKSKLSNQAKSALNSYLNIDRNHKQGNIAEVLIRVKSLVEFILEDYLNNHFLDVITYKDGKPFLNASKYPEILKKFQEDAEMRGKEYHSGYLSLPAYIGILKFFEPNHDLLKHIYKIQEINQDRNKVAHSLQAFDRKNLKKVSSAVFASKQILLASFDIDNHWFSFYEDLNQEIKKLL

SEQ ID NO: 14 - protein, 248 aa, synthetic construct
  REGION 1..248: note = Description of Artificial Sequence: Synthetic polypeptide
MIVKLRYKINLPNSLRTQNIGSTLHGVLMELLPSELVEHLHNLSYNPFRQRLIFEKELVIWEIVGLHKMVSEELLKLENLREITIKRAQKTVSLSLLSKDAIAVDDLVKKEMGREIDSRIISLKFTSPTSFKANGHYDIFPDIRKIFRSLMMNFDFFSETTKIYDYEVLSYIEENVHIVSYKLMTKNFHLEKIKVKGFQGDMTLKVTGAEQFVKLVLLMIKYATFAGIGMKTSLGMGGVSINERHYLR

SEQ ID NO: 15 - RNA, 37 nt, synthetic construct
  misc_feature 1..37: note = Description of Artificial Sequence: Synthetic oligonucleotide
  source 1..37: mol_type = other RNA
tcttcttcaggttggacagctggtgctgccaagagca

SEQ ID NO: 16 - RNA, 3822 nt, synthetic construct
  misc_feature 1..3822: note = Description of Artificial Sequence: Synthetic polynucleotide
  source 1..3822: mol_type = other RNA
ggggaattgtgagcggataacaattcccctctagaaataattttgtttaactttaagaaggagactcttctatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtg

SEQ ID NO: 17 - DNA, 54 bp, synthetic construct
  misc_feature 1..54: note = Description of Artificial Sequence: Synthetic primer
  source 1..54: mol_type = other DNA
taatacgactcactataggagttgaagcttggttcaaagaacgtatcaagagca

SEQ ID NO: 18 - DNA, 54 bp, synthetic construct
  misc_feature 1..54: note = Description of Artificial Sequence: Synthetic primer
  source 1..54: mol_type = other DNA
tgctcttgatacgttctttgaaccaagcttcaactcctatagtgagtcgtatta

SEQ ID NO: 19 - DNA, 37 bp, synthetic construct
  misc_feature 1..37: note = Description of Artificial Sequence: Synthetic primer
  source 1..37: mol_type = other DNA
ggagttgaagcttggttcaaagaacgtatcaagagca

SEQ ID NO: 20 - DNA, 37 bp, synthetic construct
  misc_feature 1..37: note = Description of Artificial Sequence: Synthetic primer
  source 1..37: mol_type = other DNA
tgctcttgatacgttctttgaaccaagcttcaactcc

SEQ ID NO: 21 - DNA, 55 bp, synthetic construct
  misc_feature 1..55: note = Description of Artificial Sequence: Synthetic primer
  source 1..55: mol_type = other DNA
taatacgactcactatagtcttcttcaggttggacagctggtgctgcagcttatt

SEQ ID NO: 22 - DNA, 55 bp, synthetic construct
  misc_feature 1..55: note = Description of Artificial Sequence: Synthetic primer
  source 1..55: mol_type = other DNA
aataagctgcagcaccagctgtccaacctgaagaagactatagtgagtcgtatta

SEQ ID NO: 23 - DNA, 55 bp, synthetic construct
  misc_feature 1..55: note = Description of Artificial Sequence: Synthetic primer
  source 1..55: mol_type = other DNA
gaaattaatacgactcactatagggaggtttcaaactttacttgctttacataga

SEQ ID NO: 24 - DNA, 30 bp, synthetic construct
  misc_feature 1..30: note = Description of Artificial Sequence: Synthetic primer
  source 1..30: mol_type = other DNA
tcctaggttgaagataacccacataataag

SEQ ID NO: 25 - DNA, 44 bp, synthetic construct
  misc_feature 1..44: note = Description of Artificial Sequence: Synthetic primer
  source 1..44: mol_type = other DNA
aggtttcaaactttacttgctttacatagaagttatttgactcc

SEQ ID NO: 26 - DNA, 20 bp, Severe acute respiratory syndrome coronavirus 2
  source 1..20: mol_type = unassigned DNA
gaccccaaaatcagcgaaat

SEQ ID NO: 27 - DNA, 24 bp, Severe acute respiratory syndrome coronavirus 2
  source 1..24: mol_type = unassigned DNA
tctggttactgccagttgaatctg

SEQ ID NO: 28 - DNA, 24 bp, Severe acute respiratory syndrome coronavirus 2
  source 1..24: mol_type = unassigned DNA
accccgcattacgtttggtggacc

SEQ ID NO: 29 - DNA, 20 bp, Severe acute respiratory syndrome coronavirus 2
  source 1..20: mol_type = unassigned DNA
ttacaaacattggccgcaaa

SEQ ID NO: 30 - DNA, 18 bp, Severe acute respiratory syndrome coronavirus 2
  source 1..18: mol_type = unassigned DNA
gcgcgacattccgaagaa

SEQ ID NO: 31 - DNA, 23 bp, Severe acute respiratory syndrome coronavirus 2
  source 1..23: mol_type = unassigned DNA
acaatttgcccccagcgcttcag

SEQ ID NO: 32 - DNA, 26 bp, Severe acute respiratory syndrome coronavirus 2
  source 1..26: mol_type = unassigned DNA
acaggtacgttaatagttaatagcgt

SEQ ID NO: 33 - DNA, 22 bp, Severe acute respiratory syndrome coronavirus 2
  source 1..22: mol_type = unassigned DNA
atattgcagcagtacgcacaca

SEQ ID NO: 34 - DNA, 26 bp, Severe acute respiratory syndrome coronavirus 2
  source 1..26: mol_type = unassigned DNA
acactagccatccttactgcgcttcg

SEQ ID NO: 35 - DNA, 19 bp, Homo sapiens
  source 1..19: mol_type = unassigned DNA
agatttggacctgcgagcg

SEQ ID NO: 36 - DNA, 20 bp, Homo sapiens
  source 1..20: mol_type = unassigned DNA
gagcggctgtctccacaagt

SEQ ID NO: 37 - DNA, 23 bp, Homo sapiens
  source 1..23: mol_type = unassigned DNA
ttctgacctgaaggctctgcgcg